(19) United States
(12) Patent Application Publication
(10) Pub. No.: US 2024/0042176 A1
(43) Pub. Date: Feb. 8, 2024

(54) IMPROVEMENTS TO FIXATION DEVICES

(71) Applicant: APERCURE SURGICAL LIMITED, Auckland (NZ)

(72) Inventors: Anthony Ronald John Phillips, Auckland (NZ); Bruce Mackenzie Stokes, Auckland (NZ); John Albert Windsor, Auckland (NZ)

(21) Appl. No.: 18/266,710

(22) PCT Filed: Dec. 16, 2021
(86) PCT No.: PCT/NZ2021/050224

§ 371 Date: Jun. 12, 2023

(30) Foreign Application Priority Data
Dec. 16, 2020 (NZ) 771162

(51) Int. Cl. A61M 25/02

(52) U.S. Cl. CPC A61M 25/02; A61M 2025/024; A61M 2025/0246; A61M 2025/0273; A61M 2025/028

(57) ABSTRACT

There is provided a fixation device to secure a catheter tube. The fixation device comprises a body with a proximal surface and a distal surface, a first opening in the proximal surface, a second opening in the distal surface, and a sidewall that connects the first opening and the second opening to form a through-hole. The body is configured to receive a first portion of the catheter tube. The fixation device further comprises a retainer configured to retain the first portion of the catheter tube in the channel. The body is configured such that the catheter tube extends in use from the body through the through-hole to an exit point at which a second portion of the catheter tube exits the patient's skin. The body is configured such that the sidewall is in use spaced from the exit point to create a window to a region of the patient's skin.

### 1. FIELD OF THE INVENTION

The present invention relates to improvements to fixation devices. In particular, the present invention relates to fixation devices for catheter tubes such as those used in medical applications.

### 2. BACKGROUND TO THE INVENTION

A catheter is a device used for various medical applications such as liquid removal (a drain) or to deliver medicine as part of a surgical procedure. It comprises a tube which is inserted into a surface of a patient's skin at an insertion point (which is an appropriately sized opening in the skin). For example, the insertion point may be a wound or other opening. Catheters can also be used to perform surgical procedures by facilitating surgical instruments entering the patient's body through the tube.

Catheters are often left in vivo for extended periods of time. Therefore, it is important to hold the catheter tube in place and prevent it being inadvertently pulled out of the insertion point. There are various ways the catheter can be held in place. One of the known approaches is to use a fixator device, which attaches to a surface of the patient's skin and to the catheter tube; this provides resistance to pulling of the catheter tube away from the insertion point.

Occlusion of catheter tubes is a significant problem. This can occur if a catheter tube is bent or kinked after it has exited the insertion point. Any blockage of the catheter tube is a significant limitation on its ability to function. As a result, many fixation devices direct the catheter tube at an angle of substantially 90° from the insertion point, e.g. where the catheter is normal to the patient's skin at the insertion point. However those devices are then relatively obtrusive.

In addition, because known fixator devices direct the catheter tube a relatively large height above the skin surface leads to snagging or catching on objects such as clothing or bed sheets. This can lead to pulling of the catheter tube out of the insertion point.

Furthermore, patients frequently lie or sit on fixator devices. The shape and relative height of those devices can cause discomfort for the patients.

Another problem with known devices is that the catheter tube is not secured effectively by the fixation device. The catheter tube is susceptible to catching because parts of it are exposed and/or because it is not properly held on the device. The catheter tube can be unintentionally displaced in or removed from the wound.

Another problem with existing fixator devices arises from the fact that they are attached to the skin adjacent to a wound. They therefore frequently cover or obscure the wound—which hinders inspection of that by a medical professional. Therefore, in order to inspect the wound, the device must be removed and reapplied or replaced after its removal. For example, in order to inspect and/or access the wound, the catheter tube would need to be removed from the device so the device can then be removed. A wound dressing would then need to be removed, and finally the dressing and device would need to be reapplied or replaced after inspection and/or accessing the wound. This is inefficient and wasteful.

It is an object of aspects of the technology to provide a fixation device for a catheter tube to overcome or ameliorate problems with existing fixation devices. Alternatively, it is an object to provide an improved fixation device for a catheter tube. Alternatively, it is an object to at least provide the public with a useful choice.

### 3. SUMMARY OF THE INVENTION

According to an aspect of the technology, there is provided a fixation device to secure a catheter tube relative to a surface of a patient's skin. In certain embodiments, the fixation device is configured to secure the catheter tube in a bent configuration relative to the surface of the patient's skin.

According to an aspect of the technology, there is provided a fixation device to secure a catheter tube in position relative to a surface of a patient's skin, wherein the fixation device comprises:
a body comprising:
   a proximal surface which, in use, faces towards the surface of the patient's skin;
   a distal surface which, in use, faces away from the surface of the patient's skin;
   a first opening in the proximal surface;
   a second opening in the distal surface;
   a sidewall that connects the first opening and the second opening to form a through-hole; and
wherein the body is configured to receive a first portion of the catheter tube,
wherein the fixation device further comprises a retainer configured to retain the first portion of the catheter tube,
wherein the body is configured such that the catheter tube extends in use from the body through the through-hole to an exit point at which a second portion of the catheter tube exits the patient's skin, and
wherein the body is configured such that the sidewall is in use spaced from the exit point to create a window to a region of the patient's skin.

In an embodiment, the fixation device may be configured to secure the catheter tube in a bent configuration relative to the surface of the patient's skin.

In an embodiment, the orientation of the first portion of the catheter tube may be substantially different to an orientation of the second portion of the catheter tube.

In an embodiment, the sidewall may substantially surround the through-hole.

In an embodiment, the body may comprise a channel formed in the distal surface which, in use, receives the first portion of the catheter tube. The channel may be configured such that a distal edge of the first portion of the catheter tube is flush with or recessed from the distal surface when the first portion of the catheter tube is positioned in the channel.

In an embodiment, the body is configured such that the catheter tube extends in use from the channel through the through-hole to the exit point.

In an embodiment, the channel may be a first channel which may, in use, receive a first portion of the catheter tube. The body may further comprise a second channel formed in the distal surface which, in use, may receive the first portion of the catheter tube as an alternative to the first portion of the catheter tube being received in the first channel. The first channel may be configured such that the catheter tube extends away from the body in a first direction when the first portion of the catheter tube is positioned in the first channel. The second channel may be configured such that the catheter tube extends away from the body in a second direction when the first portion of the catheter tube is positioned in the second channel. The first direction may be substantially different from the second direction.

In an embodiment, the body may comprise a channel portion which is common to the first channel and to the second channel.

In an embodiment, the body is configured such that the sidewall is in use spaced from the exit point to create a window to a region of the patient's skin that substantially surrounds the exit point.

In an embodiment, the body may further comprise one or more protrusions configured to grip a dressing positioned on or proximate the exit point.

In an embodiment, the body may be configured such that, when viewed from a side in a direction substantially parallel to the proximal surface, the distal surface has a substantially convex-shaped envelope.

According to an aspect of the technology, there is provided a fixation device to secure a catheter tube in a bent configuration relative to a surface of a patient's skin, wherein the fixation device comprises:
a body comprising:
   a proximal surface which, in use, faces towards the surface of the patient's skin;
   a distal surface which, in use, faces away from the surface of the patient's skin;
   a first opening in the proximal surface;
   a second opening in the distal surface;
   a sidewall that connects the first opening and the second opening to form a through-hole, wherein the sidewall substantially surrounds the through-hole; and
   a channel formed in the distal surface which, in use, receives a first portion of the catheter tube, wherein the channel is configured such that a distal edge of the first portion of the catheter tube is flush with or recessed from the distal surface when the first portion of the catheter tube is positioned in the channel,
   wherein the fixation device further comprises a retainer configured to retain the first portion of the catheter tube in the channel,
   wherein the body is configured such that the catheter tube extends in use from the channel through the through-hole to an exit point at which a second portion of the catheter tube exits the patient's skin, wherein the orientation of the first portion of the catheter tube is substantially different to an orientation of the second portion of the catheter tube, and
   wherein the body is configured such that the sidewall is in use spaced from the exit point to create a window to a region of the patient's skin that substantially surrounds the exit point.

In an embodiment, the body may comprise a channel wall forming the channel therein. The channel wall may be configured to act as the retainer.

In an embodiment, the body may be configured such that a distal region of the channel forms a slit which extends from the distal surface to a proximal region of the channel. The slit may have a width that is smaller than a width of the first portion of the catheter tube configured to be received in the channel.

In an embodiment, the body may be configured to form a lead-in to the slit to facilitate insertion of the catheter tube into the channel in use.

In an embodiment, the retainer may comprise a clip. The clip may be permanently attached to the body. The clip may be removably attached to the body.

In an embodiment, the sidewall may be configured to form an overhang. An end of the channel may be provided in the overhang. When in use, the overhang may space the second portion of the catheter tube from a proximal region of the sidewall.

In an embodiment, the first opening in the proximal surface may be smaller than the second opening in the distal surface.

In an embodiment, the sidewall may be shaped to form a chamber around the exit point in use.

In an embodiment, the chamber may be substantially dome-shaped.

In an embodiment, the body may be configured to form a gap such that the second portion of the catheter tube can pass through the gap and into the through-hole when attaching the fixation device to the catheter tube in situ. The gap may extend from an end of the body to the side wall.

In an embodiment, the width of the gap may be substantially equal to or less than the diameter of the catheter tube.

In an embodiment, the body may be configured to be able to deform to an extent sufficient for a user to manually open the gap to pass the second portion of the catheter tube through the gap.

In an embodiment, the body may further comprise one or more protrusions configured to grip a dressing positioned on or proximate the exit point.

In an embodiment, the body may be configured such that, when viewed from a side in a direction substantially parallel to the proximal surface, the distal surface has a substantially convex-shaped envelope.

In an embodiment, the body may be configured such that, when the first portion of the catheter tube is received in the channel in use, the catheter tube exits and extends away from the body in a substantially parallel direction to the patient's skin.

In an embodiment, the channel may be a first channel which may, in use, receive a first portion of the catheter tube. The body may further comprises a second channel formed in the distal surface which, in use, may receive the first portion of the catheter tube as an alternative to the first portion of the catheter tube being received in the first channel. The first channel may be configured such that the catheter tube extends away from the body in a first direction when the first portion of the catheter tube is positioned in the first channel. The second channel may be configured such that the catheter tube extends away from the body in a second direction when the first portion of the catheter tube is positioned in the second channel. The first direction may be substantially different from the second direction.

In an embodiment, the body may comprise a channel portion which is common to the first channel and to the second channel.

In an embodiment, the body may be configured such that, when viewed in cross-section from a side in a direction substantially perpendicular to the proximal surface, the second channel comprises one or more curved regions which bend a third portion of the catheter tube.

In an embodiment, the body may be configured such that, when viewed in cross-section from a side in a direction substantially parallel to the proximal surface, the channel comprises one or more curved regions which bend the first portion of the catheter.

In an embodiment, the fixation device may further comprise a movable cover configured to cover at least a section of the window.

In an embodiment, the body may be made from a substantially resilient material.

According to an aspect of the technology, there is provided a catheter system for medical applications, wherein the catheter system comprises:
a catheter tube configured to be inserted into an exit point in a patient's skin; and
a fixation device according to any of the other aspects of the technology, or as described herein.

According to an aspect of the technology, there is provided a fixation device to secure a catheter tube in a bent configuration relative to a surface of a patient's skin, wherein the fixation device comprises:
a body comprising:
   a proximal surface which, in use, faces towards the surface of the patient's skin;
   a distal surface which, in use, faces away from the surface of the patient's skin;
   a first opening in the proximal surface;
   a second opening in the distal surface; and
   a sidewall that connects the first opening and the second opening to form a through-hole, wherein the sidewall substantially surrounds the through-hole;
wherein the body is configured to receive a first portion of the catheter tube,
wherein the fixation device further comprises a retainer configured to retain the first portion of the catheter tube to the body,
wherein the body is configured such that the catheter tube extends in use from the body through the through-hole to an exit point at which a second portion of the catheter tube exits the patient's skin, wherein the orientation of the first portion of the catheter tube is substantially different to an orientation of the second portion of the catheter tube,
wherein the body is configured such that the sidewall is in use spaced from the exit point to create a window to a region of the patient's skin that substantially surrounds the exit point, and
wherein the body further comprises one or more protrusions configured to grip a dressing positioned on or proximate the exit point.

In an embodiment, the body may comprise a channel formed in the distal surface which, in use, receives the first portion of the catheter tube. The channel may be configured such that a distal edge of the first portion of the catheter tube is flush with or recessed from the distal surface when the first portion of the catheter tube is positioned in the channel.

In an embodiment, the body is configured such that the catheter tube extends in use from the channel through the through-hole to the exit point.

In an embodiment, the body may comprise a channel wall forming the channel therein. The channel wall may be configured to act as the retainer.

In an embodiment, the body may be configured such that a distal region of the channel forms a slit which extends from the distal surface to a proximal region of the channel. The slit may have a width that is smaller than a width of the first portion of the catheter tube configured to be received in the channel.

In an embodiment, the body may be configured to form a lead-in to the slit to facilitate insertion of the catheter tube into the channel in use.

In an embodiment, the retainer may comprise a clip. The clip may be permanently attached to the body. The clip may be removably attached to the body.

In an embodiment, the sidewall may be configured to form an overhang. An end of the channel may be provided in the overhang. When in use, the overhang may space the second portion of the catheter tube from a proximal region of the sidewall.

In an embodiment, the first opening in the proximal surface may be smaller than the second opening in the distal surface.

In an embodiment, the sidewall may be shaped to form a chamber around the exit point in use.

In an embodiment, the chamber may be substantially dome-shaped.

In an embodiment, the body may be configured to form a gap such that the second portion of the catheter tube can pass through the gap and into the through-hole when attaching the fixation device to the catheter tube in situ. The gap may extend from an end of the body to the side wall.

In an embodiment, the width of the gap may be substantially equal to or less than the diameter of the catheter tube.

In an embodiment, the body may be configured to be able to deform to an extent sufficient for a user to manually open the gap to pass the second portion of the catheter tube through the gap.

In an embodiment, the body may be configured such that, when viewed from a side in a direction substantially parallel to the proximal surface, the distal surface has a substantially convex-shaped envelope.

In an embodiment, the body may be configured such that, when the first portion of the catheter tube is received in the channel in use, the catheter tube exits and extends away from the body in a substantially parallel direction to the patient's skin.

In an embodiment, the channel may be a first channel which may, in use, receive a first portion of the catheter tube. The body may further comprises a second channel formed in the distal surface which, in use, may receive the first portion of the catheter tube as an alternative to the first portion of the catheter tube being received in the first channel. The first channel may be configured such that the catheter tube extends away from the body in a first direction when the first portion of the catheter tube is positioned in the first channel. The second channel may be configured such that the catheter tube extends away from the body in a second direction when the first portion of the catheter tube is positioned in the second channel. The first direction may be substantially different from the second direction.

In an embodiment, the body may comprise a channel portion which is common to the first channel and to the second channel.

In an embodiment, the body may be configured such that, when viewed in cross-section from a side in a direction substantially perpendicular to the proximal surface, the second channel comprises one or more curved regions which bend a third portion of the catheter tube.

In an embodiment, the body may be configured such that, when viewed in cross-section from a side in a direction substantially parallel to the proximal surface, the channel comprises one or more curved regions which bend the first portion of the catheter.

In an embodiment, the fixation device may further comprise a movable cover configured to cover at least a section of the window.

In an embodiment, the body may be made from a substantially resilient material.

According to an aspect of the technology, there is provided a fixation device to secure a catheter tube in a bent configuration relative to a surface of a patient's skin, wherein the fixation device comprises:
a body comprising:
   a proximal surface which, in use, faces towards the surface of the patient's skin;
   a distal surface which, in use, faces away from the surface of the patient's skin;
   a first opening in the proximal surface;
   a second opening in the distal surface; and
   a sidewall that connects the first opening and the second opening to form a through-hole, wherein the sidewall substantially surrounds the through-hole;
wherein the body is configured to receive a first portion of the catheter tube,
wherein the fixation device further comprises a retainer configured to retain the first portion of the catheter tube to the body,
wherein the body is configured such that the catheter tube extends in use from the body through the through-hole to an exit point at which a second portion of the catheter tube exits the patient's skin, wherein the orientation of the first portion of the catheter tube is substantially different to an orientation of the second portion of the catheter tube,
wherein the body is configured such that the sidewall is in use spaced from the exit point to create a window to a region of the patient's skin that substantially surrounds the exit point, and
wherein the body is configured such that, when viewed from a side in a direction substantially parallel to the proximal surface, the distal surface has a substantially convex-shaped envelope.

In an embodiment, the body may comprise a channel formed in the distal surface which, in use, receives the first portion of the catheter tube. The channel may be configured such that a distal edge of the first portion of the catheter tube is flush with or recessed from the distal surface when the first portion of the catheter tube is positioned in the channel.

In an embodiment, the body is configured such that the catheter tube extends in use from the channel through the through-hole to the exit point.

In an embodiment, the body may comprise a channel wall forming the channel therein. The channel wall may be configured to act as the retainer.

In an embodiment, the body may be configured such that a distal region of the channel forms a slit which extends from the distal surface to a proximal region of the channel. The slit may have a width that is smaller than a width of the first portion of the catheter tube configured to be received in the channel.

In an embodiment, the body may be configured to form a lead-in to the slit to facilitate insertion of the catheter tube into the channel in use.

In an embodiment, the retainer may comprise a clip. The clip may be permanently attached to the body. The clip may be removably attached to the body.

In an embodiment, the sidewall may be configured to form an overhang. An end of the channel may be provided in the overhang. When in use, the overhang may space the second portion of the catheter tube from a proximal region of the sidewall.

In an embodiment, the first opening in the proximal surface may be smaller than the second opening in the distal surface.

In an embodiment, the sidewall may be shaped to form a chamber around the exit point in use.

In an embodiment, the chamber may be substantially dome-shaped.

In an embodiment, the body may be configured to form a gap such that the second portion of the catheter tube can pass through the gap and into the through-hole when attaching the fixation device to the catheter tube in situ. The gap may extend from an end of the body to the side wall.

In an embodiment, the width of the gap may be substantially equal to or less than the diameter of the catheter tube.

In an embodiment, the body may be configured to be able to deform to an extent sufficient for a user to manually open the gap to pass the second portion of the catheter tube through the gap.

In an embodiment, the body may further comprise one or more protrusions configured to grip a dressing positioned on or proximate the exit point.

In an embodiment, the body may be configured such that, when the first portion of the catheter tube is received in the channel in use, the catheter tube exits and extends away from the body in a substantially parallel direction to the patient's skin.

In an embodiment, the channel may be a first channel which may, in use, receive a first portion of the catheter tube. The body may further comprises a second channel formed in the distal surface which, in use, may receive the first portion of the catheter tube as an alternative to the first portion of the catheter tube being received in the first channel. The first channel may be configured such that the catheter tube extends away from the body in a first direction when the first portion of the catheter tube is positioned in the first channel. The second channel may be configured such that the catheter tube extends away from the body in a second direction when the first portion of the catheter tube is positioned in the second channel. The first direction may be substantially different from the second direction.

In an embodiment, the body may comprise a channel portion which is common to the first channel and to the second channel.

In an embodiment, the body may be configured such that, when viewed in cross-section from a side in a direction substantially perpendicular to the proximal surface, the second channel comprises one or more curved regions which bend a third portion of the catheter tube.

In an embodiment, the body may be configured such that, when viewed in cross-section from a side in a direction substantially parallel to the proximal surface, the channel comprises one or more curved regions which bend the first portion of the catheter.

In an embodiment, the fixation device may further comprise a movable cover configured to cover at least a section of the window.

In an embodiment, the body may be made from a substantially resilient material.

According to an aspect of the technology, there is provided a fixation device to secure a catheter tube in a bent configuration relative to a surface of a patient's skin, wherein the fixation device comprises:
a body comprising:
   a proximal surface which, in use, faces towards the surface of the patient's skin;
   a distal surface which, in use, faces away from the surface of the patient's skin;
   a first opening in the proximal surface;
   a second opening in the distal surface;
   a sidewall that connects the first opening and the second opening to form a through-hole, wherein the sidewall substantially surrounds the through-hole;
   a first channel formed in the distal surface and which, in use, may receive a first portion of the catheter tube; and
   a second channel formed in the distal surface and which, in use, may receive the first portion of the catheter tube as an alternative to the first portion of the catheter tube being received in the first channel,
wherein the first channel is configured such that the catheter tube extends away from the body in a first direction when the first portion of the catheter tube is positioned in the first channel, and the second channel is configured such that the catheter tube extends away from the body in a second direction when the first portion of the catheter tube is positioned in the second channel, wherein the first direction is substantially different from the second direction,
wherein the fixation device further comprises a retainer configured to retain the first portion of the catheter tube in at least one of the first channel and second channel,
wherein the body is configured such that the catheter tube extends in use from the body through the through-hole to an exit point at which a second portion of the catheter tube exits the patient's skin, wherein the orientation of the first portion of the catheter tube is substantially different to an orientation of the second portion of the catheter tube, and
wherein the body is configured such that the sidewall is in use spaced from the exit point to create a window to a region of the patient's skin that substantially surrounds the exit point.

In an embodiment, the body may comprise a channel wall forming the channel therein. The channel wall may be configured to act as the retainer.

In an embodiment, the body may be configured such that a distal region of the channel forms a slit which extends from the distal surface to a proximal region of the channel. The slit may have a width that is smaller than a width of the first portion of the catheter tube configured to be received in the channel.

In an embodiment, the body may be configured to form a lead-in to the slit to facilitate insertion of the catheter tube into the channel in use.

In an embodiment, the retainer may comprise a clip. The clip may be permanently attached to the body. The clip may be removably attached to the body.

In an embodiment, the sidewall may be configured to form an overhang. An end of the channel may be provided in the overhang. When in use, the overhang may space the second portion of the catheter tube from a proximal region of the sidewall.

In an embodiment, the first opening in the proximal surface may be smaller than the second opening in the distal surface.

In an embodiment, the sidewall may be shaped to form a chamber around the exit point in use.

In an embodiment, the chamber may be substantially dome-shaped.

In an embodiment, the body may be configured to form a gap such that the second portion of the catheter tube can pass through the gap and into the through-hole when attaching the fixation device to the catheter tube in situ. The gap may extend from an end of the body to the side wall.

In an embodiment, the width of the gap may be substantially equal to or less than the diameter of the catheter tube.

In an embodiment, the body may be configured to be able to deform to an extent sufficient for a user to manually open the gap to pass the second portion of the catheter tube through the gap.

In an embodiment, the body may further comprise one or more protrusions configured to grip a dressing positioned on or proximate the exit point.

In an embodiment, the body may be configured such that, when viewed from a side in a direction substantially parallel to the proximal surface, the distal surface has a substantially convex-shaped envelope.

In an embodiment, the body may be configured such that, when the first portion of the catheter tube is received in the channel in use, the catheter tube exits and extends away from the body in a substantially parallel direction to the patient's skin.

In an embodiment, the body may comprise a channel portion which is common to the first channel and to the second channel.

In an embodiment, the body may be configured such that, when viewed in cross-section from a side in a direction substantially perpendicular to the proximal surface, the second channel comprises one or more curved regions which bend a third portion of the catheter tube.

In an embodiment, the body may be configured such that, when viewed in cross-section from a side in a direction substantially parallel to the proximal surface, the channel comprises one or more curved regions which bend the first portion of the catheter.

In an embodiment, the fixation device may further comprise a movable cover configured to cover at least a section of the window.

In an embodiment, the body may be made from a substantially resilient material.

According to an aspect of the technology, there is provided a fixation device to secure a catheter tube in a bent configuration relative to a surface of a patient's skin, wherein the fixation device comprises:
a body comprising:
   a proximal surface which, in use, faces towards the surface of the patient's skin;
   a distal surface which, in use, faces away from the surface of the patient's skin;
   a first opening in the proximal surface;
   a second opening in the distal surface; and
   a sidewall that connects the first opening and the second opening to form a through-hole, wherein the sidewall substantially surrounds the through-hole;
wherein the body is configured to receive a first portion of the catheter tube,
wherein the fixation device further comprises a retainer configured to retain the first portion of the catheter tube to the body,
wherein the body is configured such that the catheter tube extends in use from the body through the through-hole to an exit point at which a second portion of the catheter tube exits the patient's skin, wherein the orientation of the first portion of the catheter tube is substantially different to an orientation of the second portion of the catheter tube,
wherein the body is configured such that the sidewall is in use spaced from the exit point to create a window to a region of the patient's skin that substantially surrounds the exit point, and
wherein the sidewall is shaped to form a chamber around the exit point in use.

In an embodiment, the chamber is substantially dome-shaped.

In an embodiment, the body may comprise a channel formed in the distal surface which, in use, receives the first portion of the catheter tube. The channel may be configured such that a distal edge of the first portion of the catheter tube is flush with or recessed from the distal surface when the first portion of the catheter tube is positioned in the channel.

In an embodiment, the body is configured such that the catheter tube extends in use from the channel through the through-hole to the exit point.

In an embodiment, the body may comprise a channel wall forming the channel therein. The channel wall may be configured to act as the retainer.

In an embodiment, the body may be configured such that a distal region of the channel forms a slit which extends from the distal surface to a proximal region of the channel. The slit may have a width that is smaller than a width of the first portion of the catheter tube configured to be received in the channel.

In an embodiment, the body may be configured to form a lead-in to the slit to facilitate insertion of the catheter tube into the channel in use.

In an embodiment, the retainer may comprise a clip. The clip may be permanently attached to the body. The clip may be removably attached to the body.

In an embodiment, the sidewall may be configured to form an overhang. An end of the channel may be provided in the overhang. When in use, the overhang may space the second portion of the catheter tube from a proximal region of the sidewall.

In an embodiment, the first opening in the proximal surface may be smaller than the second opening in the distal surface.

In an embodiment, the body may be configured to form a gap such that the second portion of the catheter tube can pass through the gap and into the through-hole when attaching the fixation device to the catheter tube in situ. The gap may extend from an end of the body to the side wall.

In an embodiment, the width of the gap may be substantially equal to or less than the diameter of the catheter tube.

In an embodiment, the body may be configured to be able to deform to an extent sufficient for a user to manually open the gap to pass the second portion of the catheter tube through the gap.

In an embodiment, the body may further comprise one or more protrusions configured to grip a dressing positioned on or proximate the exit point.

In an embodiment, the body may be configured such that, when viewed from a side in a direction substantially parallel to the proximal surface, the distal surface has a substantially convex-shaped envelope.

In an embodiment, the body may be configured such that, when the first portion of the catheter tube is received in the channel in use, the catheter tube exits and extends away from the body in a substantially parallel direction to the patient's skin.

In an embodiment, the channel may be a first channel which may, in use, receive a first portion of the catheter tube. The body may further comprises a second channel formed in the distal surface which, in use, may receive the first portion of the catheter tube as an alternative to the first portion of the catheter tube being received in the first channel. The first channel may be configured such that the catheter tube extends away from the body in a first direction when the first portion of the catheter tube is positioned in the first channel. The second channel may be configured such that the catheter tube extends away from the body in a second direction when the first portion of the catheter tube is positioned in the second channel. The first direction may be substantially different from the second direction.

In an embodiment, the body may comprise a channel portion which is common to the first channel and to the second channel.

In an embodiment, the body may be configured such that, when viewed in cross-section from a side in a direction substantially perpendicular to the proximal surface, the second channel comprises one or more curved regions which bend a third portion of the catheter tube.

In an embodiment, the body may be configured such that, when viewed in cross-section from a side in a direction substantially parallel to the proximal surface, the channel comprises one or more curved regions which bend the first portion of the catheter.

In an embodiment, the fixation device may further comprise a movable cover configured to cover at least a section of the window.

In an embodiment, the body may be made from a substantially resilient material.

According to an aspect of the technology, there is provided a fixation device to secure a catheter tube in position relative to a surface of a patient's skin, wherein the fixation device comprises:
a body which has a proximal surface which in use faces towards the surface of the patient's skin, a distal surface, and a window,
wherein the window is defined by a first opening in the proximal surface, a second opening in the distal surface and a sidewall that connects the first opening and the second opening;
wherein in use the first portion of the catheter tube exits from the patient's skin and extends into the window,
wherein the sidewall is structured to create an overhang which provides a gap between the first portion of the catheter tube and the sidewall; and
a retainer configured to in use retain a portion of the catheter tube to the body.

In an embodiment, the catheter tube comprises a first portion, second portion and a third portion. In an embodiment, the fixation device may be configured to hold the catheter tube in a bent configuration having the first portion in between the second portion and the third portion, and wherein in the bent configuration the third portion lies substantially parallel to the patient's skin. The first portion may be a bent region.

In an embodiment, the bent region may provide an approximately 90° angle between the second portion and the third portion.

In an embodiment, the body may comprise a curved bearing surface against which the bent region lies in use.

In an embodiment, the catheter tube may have a bend radius, and further wherein the bearing surface may curve about a radius which is equal to or less than the bend radius.

Throughout the present specification, reference to the term “bend radius” should be understood as meaning the minimum radius that the catheter tube can be bent without being supported at a bent region thereof, i.e. an unsupported catheter tube, before the catheter tube kinks, buckles and/or occludes.

In an embodiment, the fixation device may further comprise a retainer configured to provide a force to the catheter tube to reduce or eliminate buckling of the catheter tube in the bent region.

In an embodiment, the side wall may comprise a first sidewall section and a second sidewall section, wherein the second sidewall section extends from the proximal surface to a proximal edge of the first sidewall section, and the first sidewall section extends from a distal edge of the second sidewall section to the distal surface, and wherein the second sidewall section is substantially straight and the first sidewall section is curved.

In an embodiment, the fixation device may comprise a cover which extends at least partially across the window.

According to another aspect of the technology, there is provided a fixation device for a catheter tube which has a bend radius, wherein the fixation device comprises:
a body which has a proximal surface which in use faces towards a surface of a patient's body and a distal surface;
a retainer which can hold the catheter tube in a bent configuration in which the catheter tube has a bent region with a curved shape which is equal to or less than the bend radius and applies a biasing force to reduce or eliminate buckling of the catheter tube in the bent region.

In an embodiment, the body may comprise a curved bearing surface against which the bent region lies in use.

In an embodiment, at least a portion of the curved bearing surface may define a concave down shape against which the bent region lies in use.

In an embodiment, the curved bearing surface may curve about a radius that is equal to or less than the bend radius.

In an embodiment, the bent region may be a first portion of the catheter tube. The catheter tube may comprise a second portion and a third portion. The bent region may provide an approximately 90° angle between the second portion of the catheter tube which exits from the patient's skin and the third region which extends substantially parallel to the patient's skin.

In an embodiment, the retainer may be provided by a channel formed in the body, and further wherein the curved bearing surface may provide a surface of the channel.

In an embodiment, the body may comprise an elongate slit configured to facilitate insertion of the catheter tube into the channel.

In an embodiment, the channel may have a diameter which is equal to or smaller than a diameter of the catheter tube to provide the biasing force.

In an embodiment, one or more portions of the channel may be at least substantially enclosed to provide the biasing force to the catheter tube.

In an embodiment, the fixation device may further comprise a retainer configured to secure the catheter tube in the channel.

In an embodiment, the fixation device may comprise a first exit pathway and a second exit pathway, wherein when the catheter tube is disposed in the first exit pathway it extends from the body in a first direction and when the catheter tube is disposed in the second exit pathway it extends away from the body in a second direction.

In an embodiment, the fixation device may further comprise a third exit pathway, and wherein when the catheter tube is disposed in the third exit pathway it extends away from the body in a third direction.

In an embodiment, at least one of the second exit pathway and the third exit pathway may each comprise a channel dimensioned to apply a biasing force to reduce or eliminate buckling of a portion of the catheter tube disposed in the exit pathway(s).

In an embodiment, the body may comprise a gap which extends from a side edge of the body to a/the sidewall to facilitate attaching the fixation device to the catheter tube in situ without removal of the catheter tube from the patient's skin.

According to another aspect of the technology, there is provided a fixation device to secure a catheter tube in position relative to a surface of a patient's skin, wherein the catheter tube has bend radius, a first portion a second portion, and a third portion, wherein the fixation device comprises:
a body which has a proximal surface which in use faces towards the surface of the patient's skin, a distal surface, and a window,
wherein the window is defined by a first opening in the proximal surface, a second opening in the distal surface and a sidewall that connects the first opening and the second opening;
wherein in use the second portion of the catheter tube exits from the patient's skin and extends into the window,
wherein the sidewall is structured to create an overhang which provides a gap between the first portion of the catheter tube and the sidewall; and
a retainer which can hold the first portion of the catheter tube in a bent configuration in which the catheter tube has a bent region with a curved shape which is equal to or less than the bend radius, and wherein the retainer applies a biasing force to reduce or eliminate buckling of the catheter tube in the bent region.

According to another aspect of the technology, there is provided a catheter arrangement for medical applications, wherein the catheter arrangement comprises:
a catheter tube configured to be inserted into an insertion point in a patient's skin, wherein the catheter tube has a bend radius; and
a fixation device as described herein.

In an embodiment, the window is provided by a through-hole which extends through the body from one side of the body to the other e.g. from a/the proximal surface to a/the distal surface.

In an embodiment, the side wall may extend around the periphery of the window.

In an embodiment, the window may be shaped and dimensioned to permit at least one of inspection of and access to, a region of the surface of the patient's body surrounding a point at which the catheter tube's exits the surface of the patient's body.

In an embodiment, the dimensions of at least one of the first opening and the second opening are relatively large compared to the diameter of the catheter tube.

In an embodiment, the dimensions of the first opening are relatively large compared to the diameter of the catheter tube to provide a relatively large separation between the edges of the first opening and the catheter tube in use.

In an embodiment, the dimensions of the first opening are larger than the dimensions of the second opening.

In an embodiment, the overhang may be configured to form the movable cover.

In an embodiment, the bearing surface may comprise a first region and a second region.

In an embodiment, the first region may extend away from an edge of the window to the second region.

In an embodiment, the second region may extend away from the first region.

In an embodiment, the first region may curve about a first radius.

In an embodiment, the second region may curve about a second radius.

In an embodiment, the first radius may be equal to the second radius.

In an embodiment, the first radius may be different from the second radius.

In an embodiment, at least one of the first radius and the second radius may be equal to or less than the bend radius of the catheter tube.

In an embodiment, the bearing surface may comprise a third region which is shaped to direct the catheter tube to lie substantially parallel to the patient's skin.

In an embodiment, the third region may curve in a different direction to one or more of the first region and the second region.

In an embodiment, the third region may extend away from the second region.

In an embodiment, the first sidewall section may extend from the proximal edge of the first region of the bearing surface to the distal edge of the second sidewall section to form an underside of the first region.

In an embodiment, the second sidewall section may extend from the proximal surface to the proximal edge of first sidewall region.

In an embodiment, the shape of the first sidewall section may be different from the second sidewall section.

In an embodiment, the fixation device may be configured to create hoop stress in a catheter tube wall when the catheter tube is secured in the channel.

In an embodiment, the biasing force is a radial force that creates hoop stress in a catheter tube wall.

In an embodiment, the retainer may be configured to provide the biasing force inwardly on the bent region of the catheter tube.

In an embodiment, the retainer may be configured to compress an outer surface of the bent region of the catheter tube.

In an embodiment, the fixation device may comprise one or more exit pathways.

In an embodiment, the body may comprise a pair of arms which each have a free end.

In an embodiment, the body may comprise a gap which extends from a side edge of the body to the window to facilitate positioning a catheter tube in the window in situ without removal of the catheter tube.

In an embodiment, the gap is provided between the free ends of the pair of arms.

Further aspects of the invention, which should be considered in all its novel aspects, will become apparent to those skilled in the art upon reading of the following description which provides at least one example of a practical application of the invention.

### 4. BRIEF DESCRIPTION OF THE DRAWINGS

The invention shall now be described by way of reference to the following non-limiting drawings, in which:

FIG. 1 is a perspective view of a fixation device and catheter tube according to an aspect of the present technology;

FIG. 2 is a first side view of a fixation device according to one form of the present technology;

FIG. 3 shows a cross-sectional view through section A-A of the fixation device shown in FIG. 2;

FIG. 4 is a second side view of the fixation device of FIG. 1;

FIG. 5 shows a cross-sectional view through section B-B of the fixation device shown in FIG. 4;

FIG. 6 is a perspective view of a fixation device for a catheter tube according to an aspect of the present technology;

FIG. 7 is a top view of the fixation device of FIG. 6;

FIG. 8 is a first side view of the fixation device of FIG. 6;

FIG. 9 shows a cross-sectional view through section A-A of the fixation device shown in FIG. 8;

FIG. 10 shows a cross-sectional view through section C-C of the fixation device shown in FIG. 8;

FIG. 11 shows an enlarged view of section D of the fixation device shown in FIG. 10;

FIG. 12 is a second side view of the fixation device of FIG. 6;

FIG. 13 is a third side view of the fixation device of FIG. 6;

FIG. 14 is a perspective view of the fixation device of FIG. 1 without the catheter tube, showing a clip in a closed position;

FIG. 15 is an enlarged view of section C of the fixation device shown in FIG. 14;

FIG. 16 is a perspective view of the fixation device of FIG. 14 showing the clip in an open position;

FIG. 17 is an enlarged view of section C of the fixation device shown in FIG. 16;

FIG. 18 is a perspective view of the fixation device of FIG. 1 holding a catheter tube, wherein the fixation device directs the catheter tube away from it in a second direction;

FIG. 19 is a first side view of the fixation device of FIG. 18;

FIG. 20 shows a cross-sectional view through section A-A of the fixation device shown in FIG. 19;

FIG. 21 is a second side view of the fixation device of FIG. 18;

FIG. 22 is a perspective view of a fixation device and catheter tube according to another aspect of the present technology;

FIG. 23 is a first side view of the fixation device of FIG. 22;

FIG. 24 shows a cross-sectional view through section A-A of the fixation device shown in FIG. 23;

FIG. 25 shows a perspective view of the cross-section through section A-A of the fixation device shown in FIG. 23;

FIG. 26 is a second side view of the fixation device of FIG. 22;

FIG. 27 shows a cross-sectional view through section B-B of the fixation device shown in FIG. 26;

FIG. 28 is a perspective view of a fixation device of FIG. 22 without a cover, showing the clip attached to the body;

FIG. 29 is a perspective view of a fixation device for a catheter tube according to an aspect of the present technology;

FIG. 30 is a top view of the fixation device of FIG. 29;

FIG. 31 is a first side view of the fixation device of FIG. 29;

FIG. 32 shows a cross-sectional view through section C-C of the fixation device shown in FIG. 31;

FIG. 33 shows a cross-sectional view through section D-D of the fixation device shown in FIG. 31;

FIG. 34 is a second side view of the fixation device of FIG. 29;

FIG. 35 shows a cross-sectional view through section A-A of the fixation device shown in FIG. 34;

FIG. 36 shows a perspective view of the cross-section through section A-A of the fixation device shown in FIG. 34;

FIG. 37A shows another first side view of the fixation device shown in FIG. 29;

FIG. 37B shows a cross-sectional view through section E-E of the fixation device shown in FIG. 37A;

FIG. 37C shows a cross-sectional view through section F-F of the fixation device shown in FIG. 37A;

FIG. 37D shows a cross-sectional view through section G-G of the fixation device shown in FIG. 37A;

FIG. 38 is a perspective view of a fixation device for a catheter tube according to an aspect of the present technology;

FIG. 39 is a top view of the fixation device of FIG. 38;

FIG. 40 is a bottom view of the fixation device of FIG. 38;

FIG. 41 shows a first side view of the fixation device shown in FIG. 38;

FIG. 42 shows a cross-sectional view through section D-D of the fixation device shown in FIG. 41;

FIG. 43 shows a second side view of the fixation device shown in FIG. 38;

FIG. 44 shows a cross-sectional view through section A-A of the fixation device shown in FIG. 43.

FIG. 45 is a perspective view of a fixation device for a catheter tube according to an aspect of the present technology;

FIG. 46 is a top view of the fixation device of FIG. 45;

FIG. 47 shows a first side view of the fixation device of FIG. 45 including a catheter tube;

FIG. 48 shows a cross-sectional view through section I-I of the fixation device and catheter tube shown in FIG. 47;

FIG. 49 shows a first side view of the fixation device of FIG. 45;

FIG. 50 shows a cross-sectional view through section G-G of the fixation device shown in FIG. 49, including a catheter tube;

FIG. 51 shows a second side view of the fixation device of FIG. 45;

FIG. 52 shows a cross-sectional view through section O-O of the fixation device shown in FIG. 51;

FIG. 53 shows an enlarged cross-sectional view through section P of the fixation device shown in FIG. 52;

FIG. 54 is another perspective view of the fixation device of FIG. 45; and

FIG. 55 shows an enlarged view of section R of the fixation device shown in FIG. 54.

### 5. DETAILED DESCRIPTION OF THE INVENTION

### 5.1 Catheter Arrangement

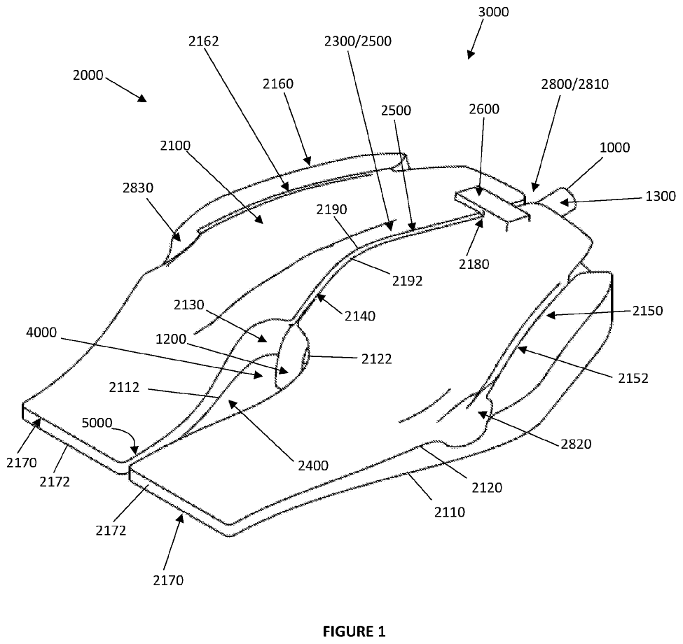

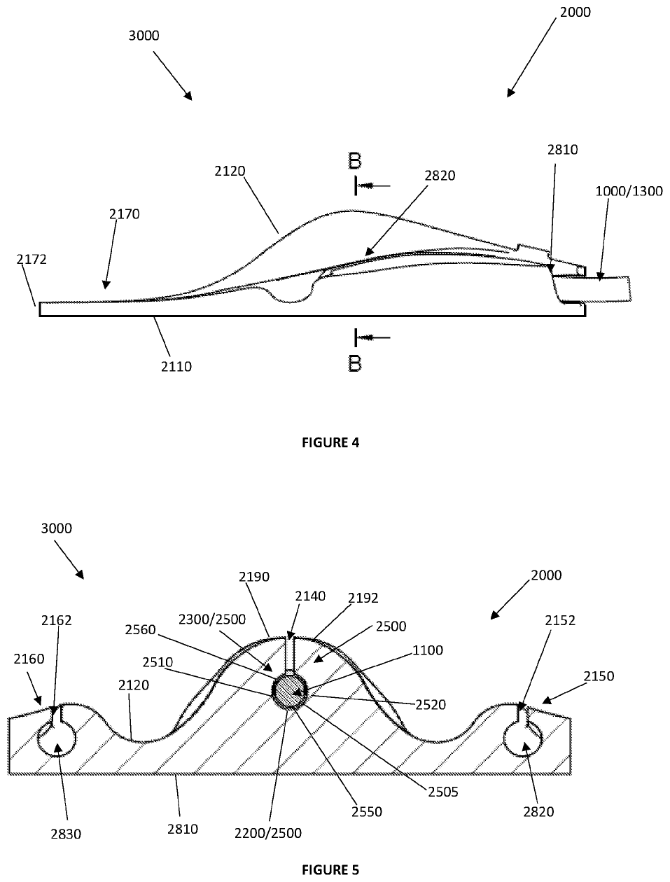

FIGS. 1 to 5 show a catheter arrangement 3000 according to an aspect of the present technology. The catheter arrangement 3000 generally comprises a catheter tube 1000 and a fixation device 2000.

The catheter tube 1000 is configured to be inserted into an exit point S1 in a patient's skin S (as is indicated in FIG. 3).

### 5.2 Fixation Device

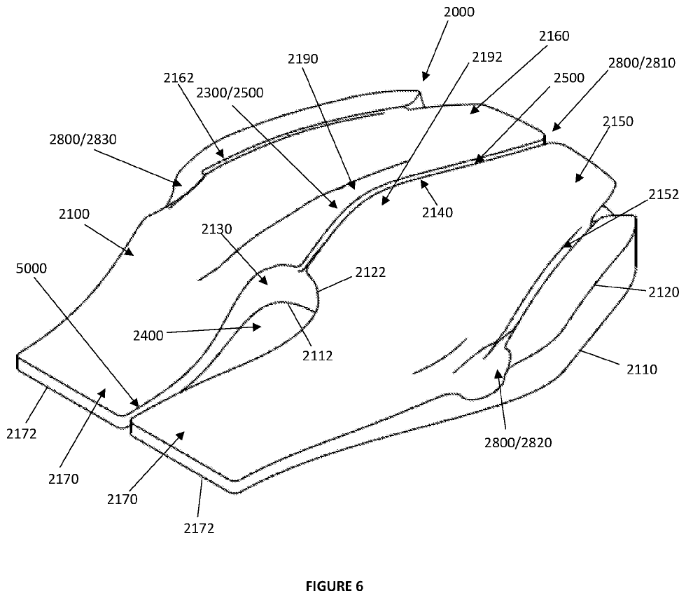

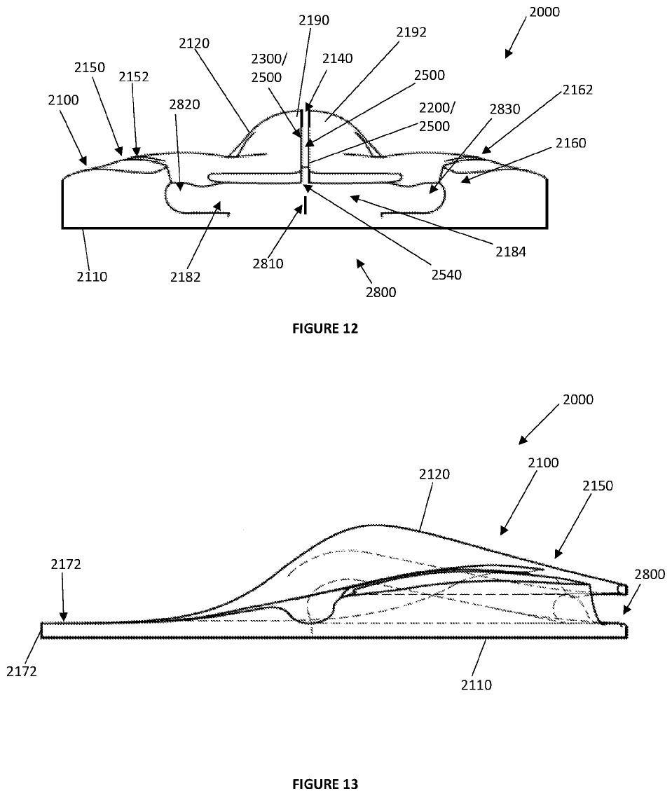

FIGS. 6 to 13 show various views of the fixation device 2000 shown in FIGS. 1 to 5, without the catheter tube 1000 attached.

The fixation device 2000 may be configured to secure the catheter tube 1000 in position relative to a surface of the patient's skin S. In the preferred forms illustrated, the fixation device 2000 is configured to secure the catheter tube 100 in a bent configuration relative to a surface of the patient's skin S.

In the bent configuration, the catheter tube 1000 has a bent region with a curved shape and the orientation of a first portion 1100 of the catheter tube 1000 may be substantially different to an orientation of a second portion 1200 of the catheter tube 1000 which exits from the patient's skin S at exit point S1.

The fixation device 2000 comprises a body 2100 which is configured to receive the first portion 1100. The body 2100 may be configured, e.g. shaped, to provide a bearing surface 2200 against which the first portion 1100 is retained against in use.

The fixation device 2000 comprises a retainer 2300 which is configured to retain the first portion 1100 to the body 2100. The retainer 2300 may retain the catheter tube 1000 against the bearing surface 2200. The retainer 2300 may be configured to retain the catheter tube 1000 in the bent configuration, as illustrated in FIG. 3.

The bearing surface 2200 and retainer 2300 may be provided by a channel 2500 formed in the body 2100.

The body 2100 comprises a proximal surface 2110 which, in use, is orientated to face towards a surface of a patient's skin S, and a distal surface 2120 which, in use, is orientated to face away from the surface of the patient's skin S.

The body 2100 may comprise a first opening 2112 in the proximal surface 2110, a second opening 2122 in the distal surface 2120, and a side wall 2130 that connects the first opening 2112 and the second opening 2122 to form a through-hole 2400. The through-hole 2400 extends through the body 2100 from the proximal surface 2110 to the distal surface 2120.

The body 2100 may be configured to space the sidewall 2130 from the exit point S1 to create a window 4000 to a region of the patient's skin. This region of the patient's skin may at least partially surround the exit point S1, and preferably it substantially or entirely surrounds the exit point S1.

These and other aspects of the fixation device 2000 will be discussed in more detail below.

### 5.2.1 Bearing Surface

The body 2100 may be configured to provide a bearing surface 2200 against which the first portion 1100 of the catheter tube 1000 is retained in use.

The catheter tube comprises the first portion 1100, the second portion 1200, and a third portion 1300 which exits the body 2100 and extends away from it. The fixation device 2000 may be configured to hold the catheter tube 1000 in a bent configuration such that an angle between the third portion 1300 and the second portion 1200 of the catheter tube 1000 is substantially 90°, as illustrated in FIG. 3. The shape of the bearing surface 2200 which defines the shape of the bent region is discussed in more detail below.

In alternative embodiments, the fixation device 2000 may be configured to hold the catheter tube 1000 in a bent configuration such that an angle between the second portion 1200 and the third portion 1300 of the catheter tube 1000 is less or more than 90°.

As illustrated in FIG. 3, the second portion 1200 extends from a surface of the patient's skin S, e.g. at an angle which is substantially perpendicular to the patient's skin S.

In the bent configuration, the third portion 1200 exits the fixation device 2000 and lies substantially parallel to the patient's skin S. The body 2100 is configured such that, when the first portion 1100 of the catheter tube 1000 is received in the channel 2500 in use, the third portion 1300 of the catheter tube 1000 exits and extends away from the body 2100 in a substantially parallel direction to the patient's skin S. This orientation may reduce or prevent the third portion 1300 from snagging/catching on other objects or surfaces.

As shown, the body 2100 is configured to allow the second portion 1200 and the third portion 1300 to be located substantially perpendicular to each other in use, e.g. with an approximately 90° degree angle between their respective axes.

In preferred forms, as shown in FIG. 3, the body 2100 is configured such that, when the first portion 1100 of the catheter tube 1000 is received in the channel 2500 in use, the catheter tube exits and extends away from the body 2100 in a substantially parallel direction to the patient's skin S.

The bearing surface 2200 is curved about a radius selected according to the bend radius of the catheter tube 1000 with which the fixation device 2000 is configured to be used.

The bearing surface 2200 may curve about a radius which is equal to or less than the bend radius of the catheter tube 1000. As can be seen, the bearing surface 2200 comprises at least a first region 2210 and a second region 2220. The first region 2210 may curve about a first radius e.g. which is equal to or less than the bend radius of the catheter tube 1000. The second region 2220 may curve about a second radius e.g. which is equal to or less than the bend radius of the catheter tube 1000. The first and second radii may be different. However, in alternative embodiments, the bearing surface 2200 or parts of it may curve about a radius or radii which is/are greater than the bend radius of the catheter tube 1000.

The fixation device 2000 can hold the catheter tube 1000 in a bent configuration e.g. with the bent region. In some forms, the first portion 1100 may form at least part of the bent region of the catheter tube 1000. The first portion 1100 or parts thereof may have a curve radius which is equal to or less than the bend radius of the catheter tube 1000. In these forms, the retainer 2300 may be configured to apply a biasing or restricting force to create hoop stress in the walls of the catheter tube 1000, to thereby reduce or eliminate buckling of the catheter tube 1000 in the bent region 1300 (as discussed further below). However, in other forms, the first portion 1100 or parts thereof may have a curve radius which is greater than the bend radius of the catheter tube 1000.

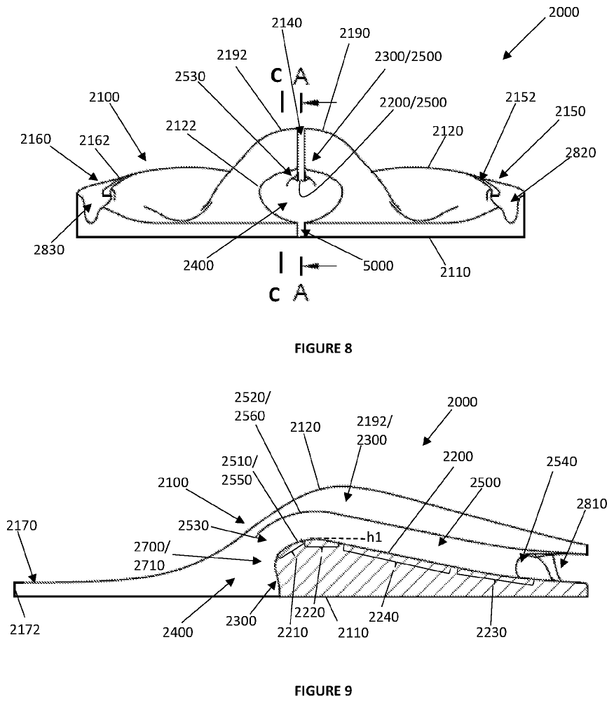

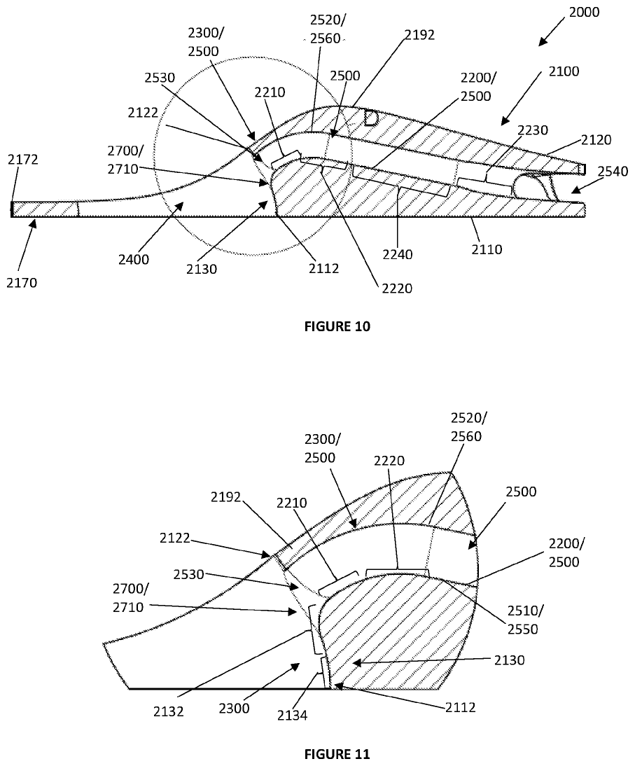

As is perhaps best shown in FIGS. 9 and 10, at least a portion of the bearing surface 2200 defines a substantially convex shape when viewed in cross-section from a direction perpendicular to the direction in which the length of the channel 2500 extends. The first region 2210 extends from the side wall 2130 to the second region 2220. The second region 2220 extends from the first region 2210 towards the second channel opening 2540. Both the first region 2210 and the second region 2220 may be convex when viewed in said cross-section. As discussed above, the first region 2210 may curve about a first radius and the second region 2220 may curve about a second radius which is different to the first radius when viewed in said cross-section. In the illustrated embodiment, the first radius may be greater than the second radius. Therefore, the amount of curvature in the first region 2210 may be greater than the amount of curvature in the second region 2220. As shown, the bearing surface 2200 flattens out as it extends towards the end of the body 2100 from the convex first and second regions 2210, 2220. The bearing surface 2200 comprises a third region 2230 which extends from an end of the body 2100 which is proximal the second channel opening 2450 towards the second channel opening 2540. The third region 2230 curves in a different direction when viewed in said cross-section to at least one of the first region 2210 and the second region 2220 e.g. the third region 2230 has a substantially concave shape. The bearing surface 2200 may have a fourth region 2240 which extends between the third region 2230 to the second region 2220. The fourth region 2240 may be a substantially straight or linear slope when viewed in said cross-section.

The body 2100 is configured such that the end of the third region 2230 proximate the end of the body 2100 is orientated approximately parallel to the proximal surface of the body 2100. The concave third region 2220 helps achieve this.

Therefore, the first region 2210, second region 2220 and third region 2230 are therefore shaped to hold a catheter tube 1000 in a bent configuration such that the second portion 1200 of the catheter tube 1000 is substantially perpendicular the patient's skin S and the third portion 1300 is substantially parallel to the patient's skin S.

The first region 2210 and the second region 2220 are contiguous with each other. In the illustrated embodiments, the third region 2230 is spaced apart from the second region 2220 by section 2240 of the bearing surface 2200, e.g. a substantially linear section. It should be appreciated that in alternative embodiments, one or more sections 2240 of the bearing surface 2200 may space apart one or more of the first, second and third regions 2210, 2220, 2230. In yet further alternative embodiments, all three of the first, second and third regions 2210, 2220, 2230 may be contiguous with each other.

The body 2100 is configured to help define the total angle that the catheter tube 1000 is bent by the fixation device 2000, e.g. the sum of the angles between bent portions of the catheter tube 1000 which are in use bent by the first, second and third regions 2210, 2220, 2230. Referring to FIG. 3, f1 is the effective angle the catheter tube 1000 is bent by the first region 2210, f2 is the effective angle the catheter tube 1000 is bent by the second region 2220, and f3 is the effective angle the catheter tube 1000 is bent by the third region. The total angle is the sum of f1+f2−f3. In the illustrated embodiment, the total angle is approximately 90°. Alternatively, the angle between the longitudinal axis A1 of the first region 1100 and the longitudinal axis A3 of the second region 1200 is approximately 90°.

### 5.2.2 Channel

According to an aspect of the present technology, the fixation device 2100 comprises a channel 2500 which, in use, receives the first portion 1100 of the catheter tube 1000.

As illustrated, the channel 2500 is formed in the distal surface 2120. The body 2100 may comprise a channel wall 2505 which forms the channel 2500 therein. At least part of the channel wall 2505 forms the bearing surface 2200, e.g. a distal surface of the channel wall 2505.

The channel 2500 may be configured such that a distal edge of the first portion 1100 of the catheter tube 1000 is flush with or recessed from the distal surface 2120 when the first portion 1100 is positioned in the channel 2500. With reference to the Figures, the distal edge is a top edge of the first portion 1100 which, in use, faces away from bearing surface 2200. Therefore, the channel 2500 may be sized to allow the first portion 1100 to sit below the distal surface 2120 in use. The depth of the channel 2500 may be the distance between a point on the distal surface 2120 of the body 2100 and a proximal surface of the channel 2500 (e.g. the bearing surface 2200). The depth of the channel 2500 may be equal to or greater than the diameter of the catheter tube 1000.

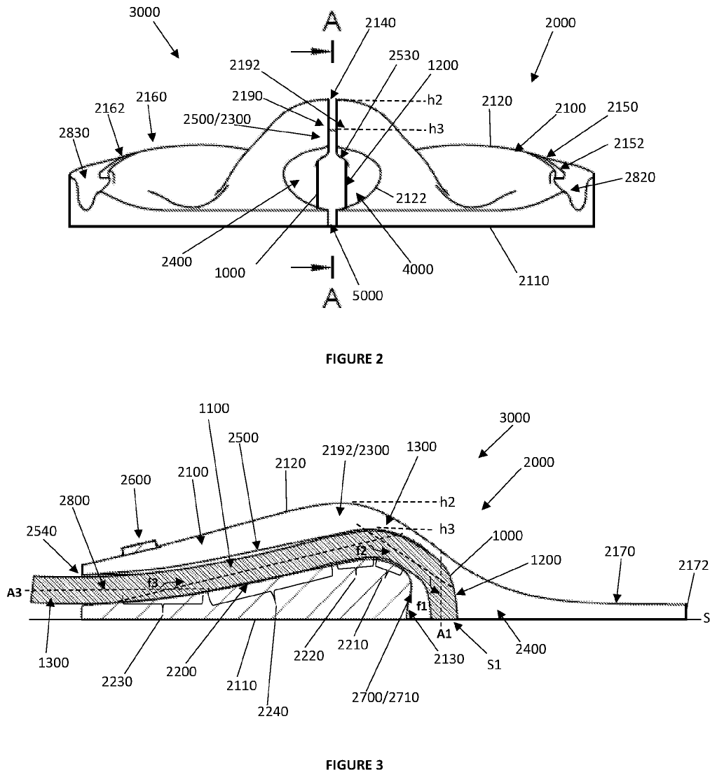

The channel 2500 may be configured to surround at least partially, and preferably substantially enclose, the first portion 1100 of the catheter tube 1000 or parts of it. As illustrated in FIGS. 2 and 5, the channel 2500 is configured to receive and at least substantially surround the first portion 1100. Therefore, the channel 2500 is substantially enclosed, for example, a distal region of the channel 2500, which may be in the form of a slit 2140 (as described below), may have a width that is less than a width of the first portion 1100 of the catheter tube 1000. This may reduce/prevent the catheter tube 1000 from snagging/catching on objects or other surfaces.

However, in alternative embodiments not shown, the channel 2500 may not be substantially enclosed, e.g. the channel 2500 could be substantially open, i.e. the distal region of the channel 2500, which may be in the form of a slit 2140, may have a width that is greater than a width of the first portion 1100 of the catheter tube 1000. In some forms, the channel 2500 is sufficiently wide to be able to accommodate catheter tubes of a range of widths. This enables the fixation device 2100 to be used with different types of catheters that may be desirable to use.

As illustrated in FIG. 3, the body 2100 is configured such that, when viewed in cross-section from a side in a direction substantially parallel to the proximal surface 2110, the channel 2500 comprises one or more curved regions (e.g. bottom regions of the channel) which bend the first portion 1100 of the catheter tube 1000.

The first portion 1100 of the catheter tube 1000 can be disposed in the channel 2500 such that it contacts the bearing surface 2200. As is perhaps best shown in FIGS. 3 and 5, the channel 2500 may retain the first portion 1100 of the catheter tube 1000 against the bearing surface 2200.

In some forms, the dimensions of the channel 2500 may help to retain the first portion 1100 of the catheter tube 1100 to the body 2100. In at least one region of the channel 2500, the distance between opposing channel wall surfaces 2510, 2520 are equal or smaller to the outer diameter of the catheter tube 1000 when that is in a straight (i.e. not bent) configuration. However, in some forms, the distance between opposing channel wall surfaces 2510, 2520, in at least a portion of the channel 2500, may be larger than the diameter of the catheter tube 1000 when that is in a straight (i.e. not bent) configuration. Furthermore, in some embodiments, the width, i.e. a distance between opposing channel wall surfaces 2510, 2520 may vary along the channel 2500. For instance, the distance between opposing channel wall surfaces 2510, 2520 in a first region of the channel 2500 which bends a first region of the catheter tube may be smaller than the distance between opposing channel wall surfaces 2510, 2520 in a second region of the channel 2500.

As illustrated, the body 2100 comprises a slit 2140 configured to facilitate insertion of the catheter tube 1000 into, and/or removal of the catheter tube 1000 from, the channel 2500. The body 2100 may be configured such that a distal region of the channel 2500 forms the slit 2140. The slit 2140 extends from the distal surface 2120 to a proximal region of the channel 2500. The slit 2140 may have a width, i.e. a distance between opposing edges of the body 2100 at the slit 2140, that is smaller than a width of the first portion 1100 of the catheter tube 1000.

At least a portion of the body 2100 forming the channel 2500 may be resiliently deformable. This may facilitate insertion and/or removal of a catheter tube from the channel 2500, e.g. pressing the catheter tube 1000 into the channel 2500.

The channel 2500 comprises a first channel opening 2530 which is orientated to face towards a first end of the body 2100, and a second channel opening 2540 which is orientated to face towards a second end of the body 2100. The channel 2500 may extend from the first channel opening 2530 to the second channel opening 2540. The first channel opening 2530 may be an opening formed in the sidewall 2130. Therefore, the channel 2500 extends from the through-hole 2400 to the second end of the body 2100.

As illustrated, the channel 2500 and/or slit 2140 may separate the body 2100 into a first side 2190 and a second side 2192. These and other aspects of the technology will be discussed in more detail below.

### 5.2.3 Retainer

According to an aspect of the present technology, the fixation device 2000 comprises a retainer 2300 which may assist with retaining the catheter tube 1000 to the body 2100. The retainer 2600 is configured to retain the catheter tube 1000 in the channel 2500 and/or on the bearing surface 2200.

In certain embodiments, the channel 2500 may retain the first portion 1100 of the catheter tube 1000. The channel wall 2505 may be configured to act as the retainer 2300. This may be achieved, for example where the channel wall 2505 substantially encloses the first portion 1100, as described earlier. For example, as is perhaps best shown in FIG. 5, the channel wall 2505 substantially surrounds the circumference of the catheter tube 1000 when it is positioned in the channel 2500.

However, in other forms, the retainer 2300 may be provided by one or more other components or arrangements. The other component(s) or arrangement(s) may be configured to retain the first portion 1100 in combination with the channel wall 2505, or as an alternative to the channel wall 2505 retaining the first portion 1100 substantially on its own. Therefore, in certain forms, the channel 2500 may receive the first portion 1100, but may not retain the first portion 1100 in the channel 2500. For instance, the retention forces may be provided by one or more other components or arrangements.

For example, in the embodiment illustrated in FIG. 1 the retainer 2300 comprises a clip 2600. In this embodiment, the retainer 2300 comprises a combination of the channel wall 2505 and the clip 2600. The clip 2600 comprises an elongate member that extends across the slit 2140, from the first side 2190 to the second side 2192. This provides resistance to the catheter tube 1000 being pulled out of the channel 2500 which helps secure it in the channel 2500.

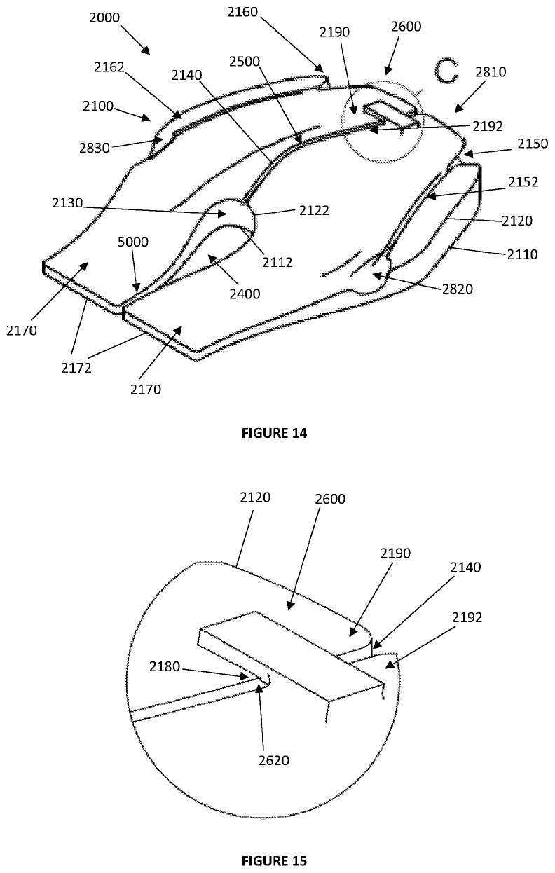

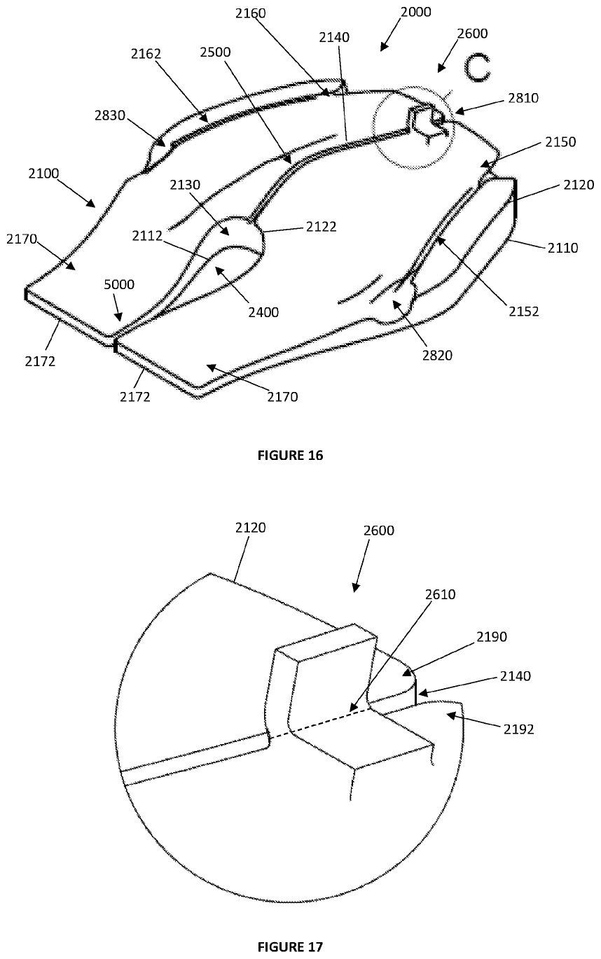

The clip 2600 can be moved between a closed position as illustrated in FIGS. 14 and 15 and an open position as shown in FIGS. 16 and 17. As illustrated in FIG. 17, the clip 2600 comprises a flexible region 2610 to permit it to move between the closed position and the open position.

The clip 2600 may also comprise a clip portion 2620 which is configured to engage with a corresponding clip portion 2180 to secure the clip 2600 in the closed position. In the open position, the clip 2600 does not hinder insertion of the catheter tube (not shown FIGS. 16 and 17) into, or removal of the catheter tube from, the channel 2500. However, in the closed position the clip 2600 provides resistance to the catheter tube 1000 being pulled out of the channel 2500.

As illustrated, the clip 2600 is formed integrally to the body 2100, i.e. it is moulded with the body 2100. In alternate embodiments, the retainer 2600 may be formed separately, and permanently or releasably attached to the body 2100.

In alternate embodiments, the retainer 2300 comprises other component(s) or arrangement(s) to secure the catheter tube 1000 in the channel 2500, e.g. one or more of a latch, a strap, a cover, adhesive, snap-fit, interference fit or other suitable fastener.

Alternatively, as in the embodiment illustrated in FIG. 6, the fixation device 2000 does not comprise a separate component, e.g. clip 2600. Instead, the channel 2500 may sufficiently enclose the catheter tube 1000, as described above, and provide sufficient frictional resistance to prevent the catheter tube 1000 from being pulled out of the channel 2500 up to a functional design limit.

In some forms, the retainer 2300 may be permanently or releasably attached to the fixation device 2000, e.g. to the body 2100.

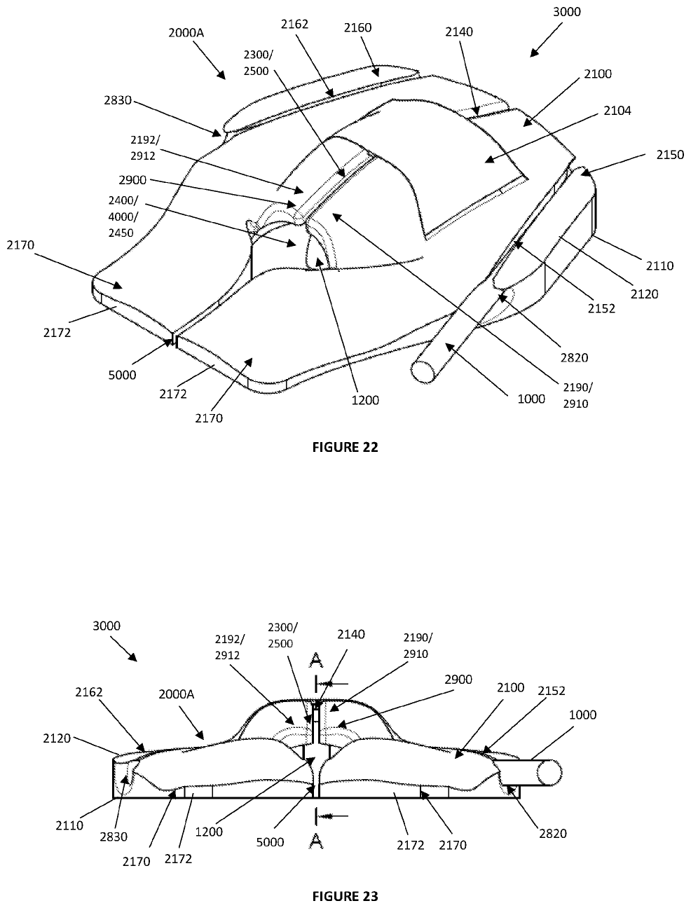

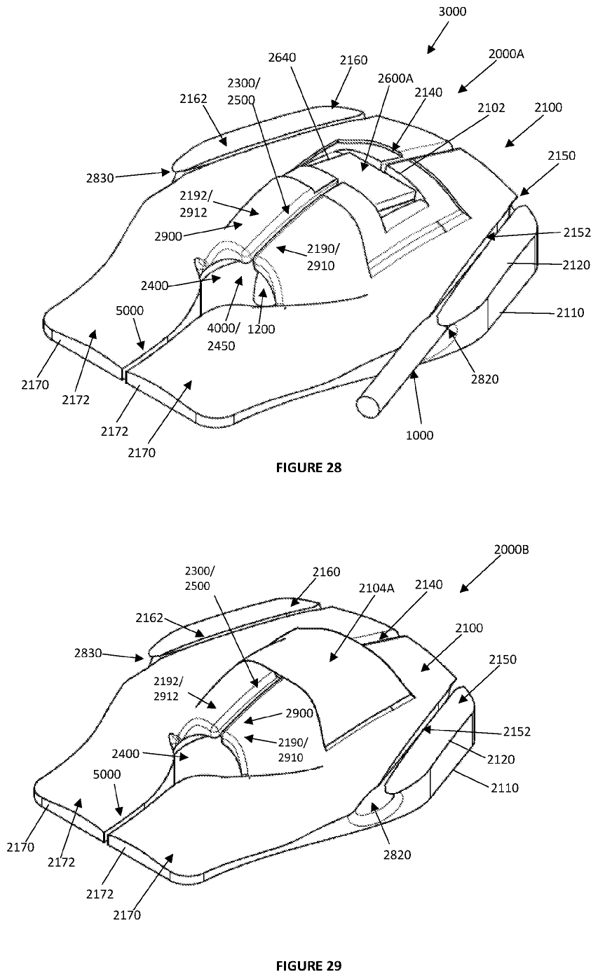

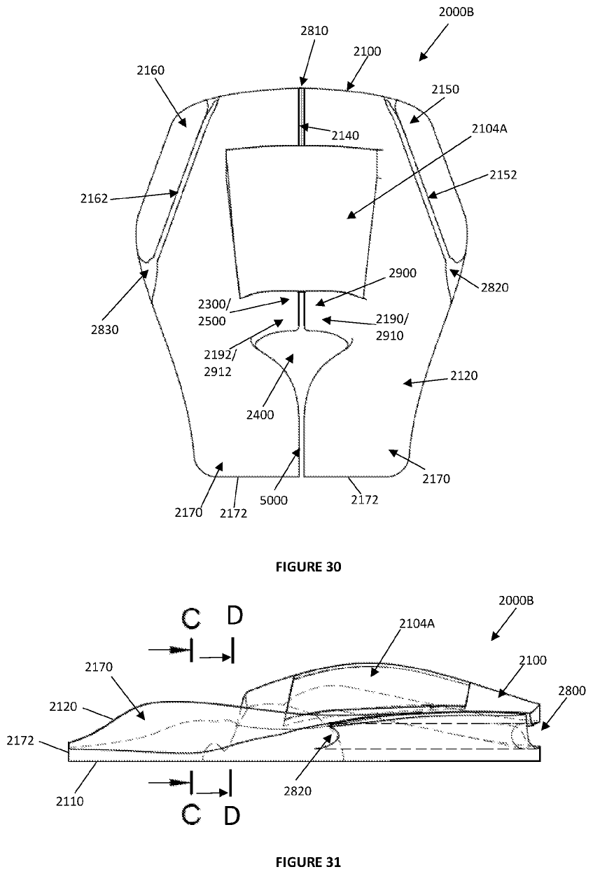

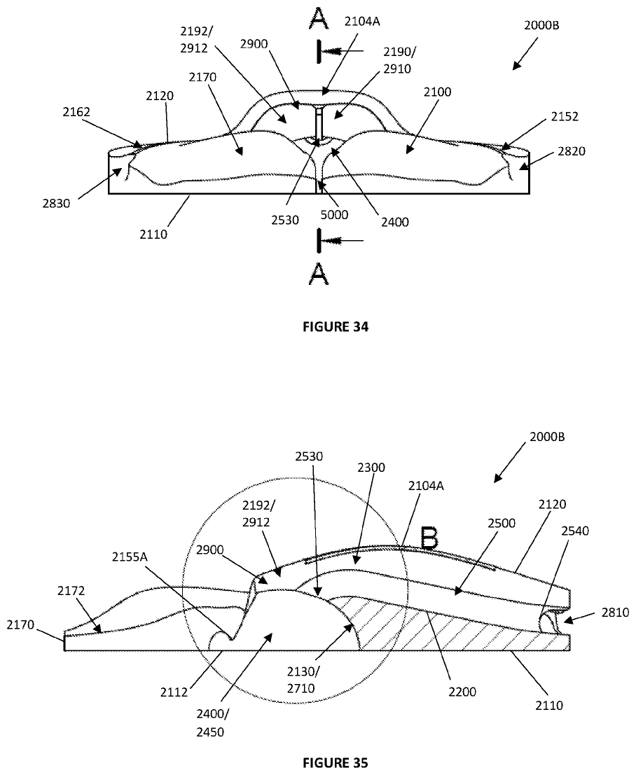

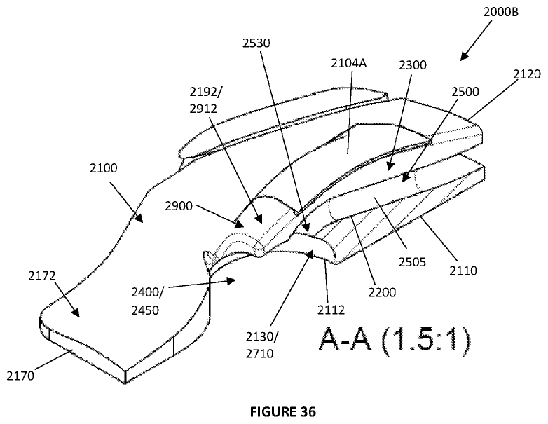

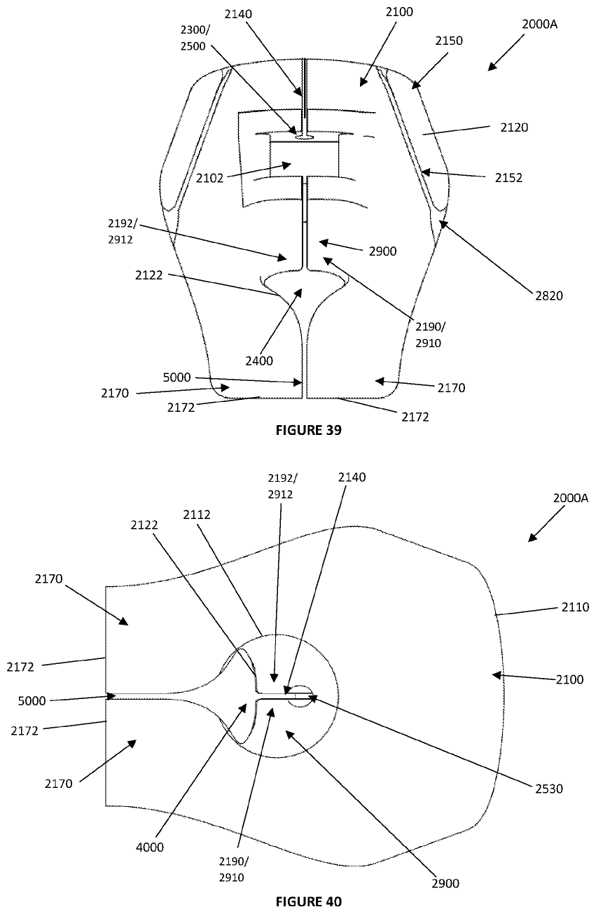

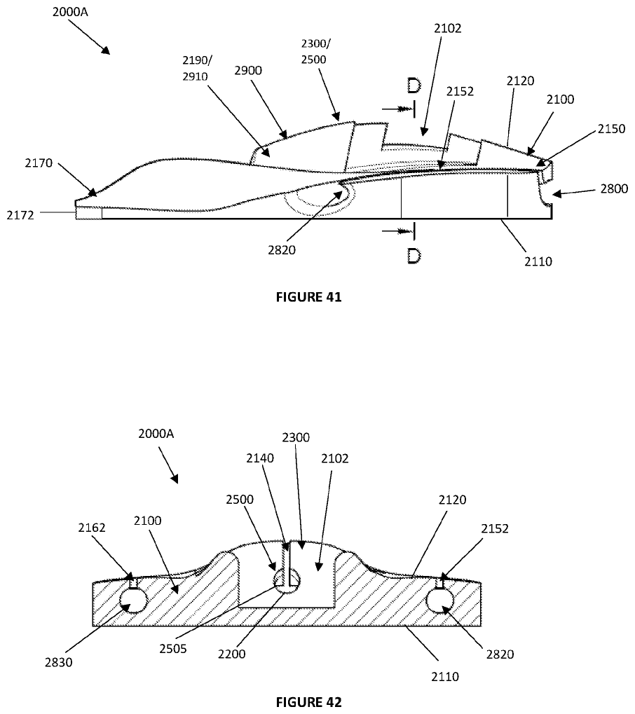

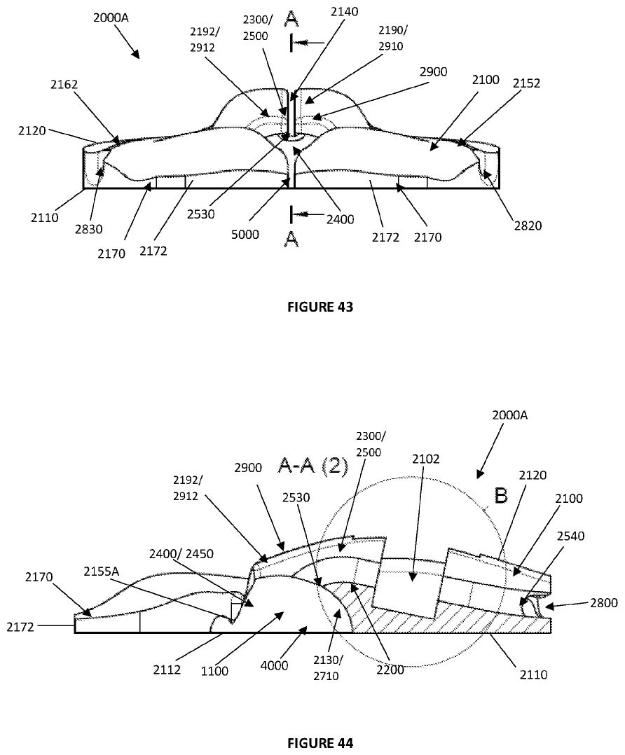

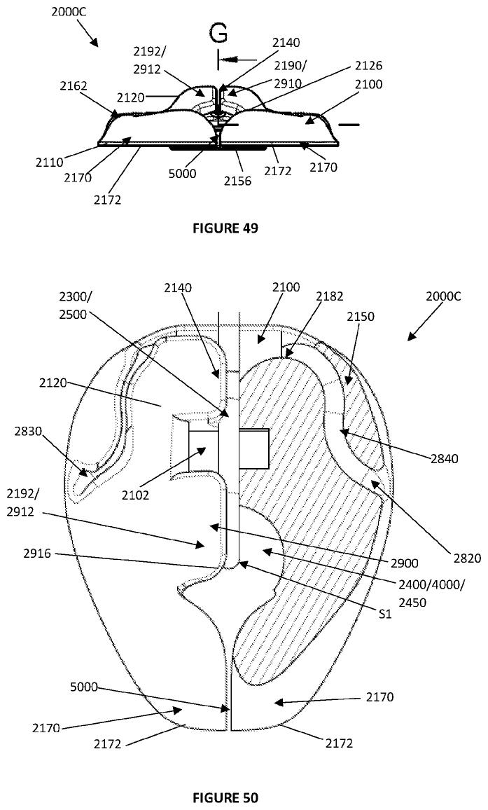

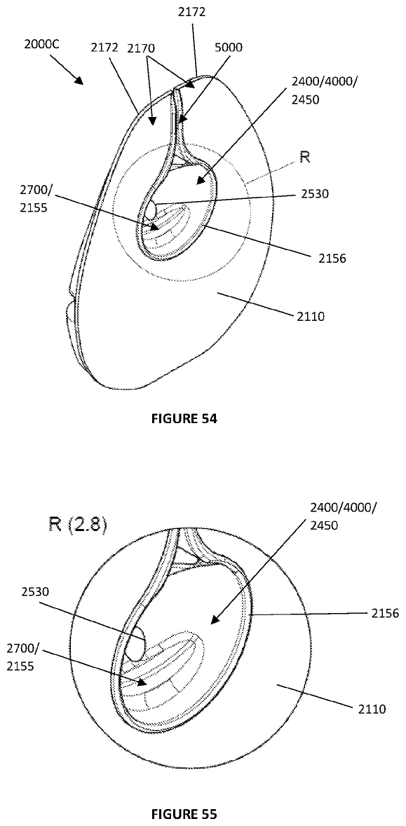

FIGS. 22 to 55 show other embodiments of a fixation device 2000A, 2000B, 2000C and catheter arrangements 3000, according to aspects of the present technology. Throughout the description and Figures like references refer to like components.

The fixation devices 2000A, 2000B, 2000C each have a different embodiment of a retainer to that described above and illustrated in at least FIGS. 1 to 5.

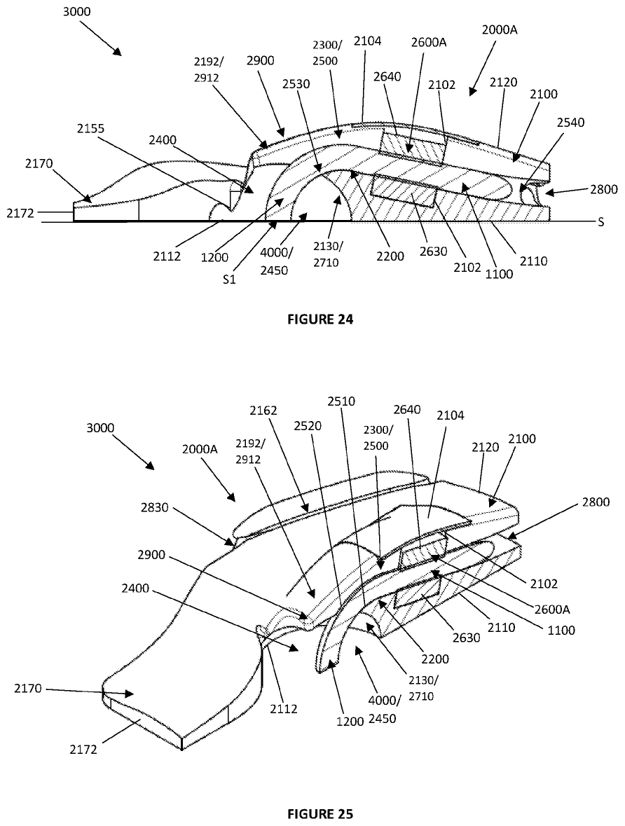

In the embodiment of FIGS. 24 to 28, the body 2100 defines a space 2102 in the region of the channel 2500 which is shaped to in use receive a clip 2600A. The embodiments shown in FIGS. 38, 39 and FIGS. 44 to 51 also have a body 2100 that defines the space 2102. In other forms, the body 2100 may define a space configured to receive in use one or more other components or arrangements to retain the catheter tube 1000 to the body 2100, as described above.

In certain forms, the clip 2600A or at least a part of it is removable from the body 2100, i.e. it is releasably attached to the body 2100. As illustrated, the entire clip 2600A is removable. However, in other forms, the clip 2600A or at least part of it may be permanently attached to the body 2100. As illustrated in FIGS. 24, 25, and FIG. 27, a first clip part 2630 interference fits with a portion of the body 2100 which defines the space 2102. The first part 2630 is configured to engage with a second clip part 2640, wherein the first and second parts 2630, 2640 are shaped to secure the catheter tube 1000 therebetween without causing it to buckle, kink or occlude. Portions of the first and second parts 2630, 2640 are shaped to receive and hold the catheter tube 1000 therebetween, e.g. the clip parts 2630, 2640 may have semi-cylindrical grooves along their surfaces which, when the clip is in place within the body 2100, receive respective parts of the catheter tube 1000. These form part of the channel 2500 which retains and holds the catheter tube 1000 in the bent configuration, and therefore, any one of more of the aspects or examples of the channel 2500 as described herein may be equally incorporated into these parts of the clip 2600A, e.g. the dimensions between opposing surfaces of the first and second clip parts 2630, 2640 may be equal to or less than the diameter of the catheter tube 1000 to provide the biasing force to the walls of the catheter tube 1000 which creates hoop stress. Therefore, clip part 2630 provides part of the bearing surface 2200 against which the first portion 1100 of the catheter tube 1000 is retained in use by the second part 2640.

As illustrated in at least FIGS. 22, 24, and 25, the retainer 2300 is in the form of a cover 2104. The cover 2104 is configured to extend across at least a portion of the space 2102, for example to extend over the entire space 2102. As can be seen, the cover 2104 may extend across the space 2102 and thereby also covers the portion of the catheter tube 100 which is disposed in the space 2102. Therefore, this embodiment comprises a clip 2600A and a cover 2104. The cover 2104 may extend over the clip 2104 so it is covered and not exposed.

The cover 2104 may be releasably attached to a part of the body 2100. This facilitates insertion of the clip 2600A (or other component) into the space 2102 or the removal therefrom.

Turning to another embodiment shown in FIGS. 29 to 37D, the body 2100 of fixation device 2000B does not include a space 2102 for receiving a clip 2600A. In this embodiment, the retainer 2600 comprises a cover 2104A which is similar to cover 2104 discussed herein. The cover 2104A extends across the slit 2140, from the first side 2190 to the second side 2192 of the body 2100. This may help reduce or prevent the first and second sides 2190, 2192 from splaying outwards, and/or may provide resistance to the first portion 1100 of the catheter tube 1000 being pulled out of the channel 2500. At least a portion of the cover 2104A may be releasably attached to a part of the body 2100. This facilitates insertion/removal of the first portion 1100 into/from the channel 2500 through the slit 2140.

Turning to yet another embodiment which is shown in FIGS. 44 to 55, the body 2100 of fixation device 2000C comprises the space 2102 for receiving a clip (not shown), but it does not include a cover, e.g. cover 2104/2104A.

In certain forms, the retainer 2300 may, as well as acting to retain the catheter tube 1000, be configured to provide a biasing or restricting force to a catheter tube 1000 to reduce or eliminate buckling or kinking when in the bent configuration. The biasing or restricting force may be directed inwardly on the catheter tube 1000 and may compress at least a portion of an outer surface of the catheter tube 1000. For instance, the biasing or restricting force is a radial force that creates hoop stress in a catheter tube wall.

### 5.2.4 Through-Hole

According to an aspect of the present technology, the fixation device 2000, 2000A, 2000B, 2000C comprises a through-hole 2400 extending between the proximal surface 2110 and the distal surface 2120 of the body 2100. The sidewall 2130 extents at least partially around the through-hole 2400. As illustrated, in preferred forms, the sidewall 2130 substantially surrounds the through-hole 2400.

In the illustrated forms, the sidewall 2130 is discontinuous and comprises a gap 5000 which extends from an end of the body 2100 to the sidewall 2130. As illustrated, the sidewall 2130 surrounds the through-hole 2400 apart from the gap 5000. The gap 5000 may be sized to be small enough to allow the sidewall 2130 to substantially surround the through-hole 2400, i.e. to a sufficient extent to achieve one or more of the advantages described herein, for example to protect the exit point and/or to retain a dressing at the exit point. For instance, the width of the gap 5000, i.e. distance between opposing edges of the body 2100 at the gap 5000, may be substantially equal to or less than the diameter of the catheter tube 1000. However, in other forms not shown, the width of the gap may be more than the diameter but still sufficiently small that the sidewall 2130 is considered to substantially surround the through-hole 2400.

The body 2100 is configured to form the gap 5000 such that the second portion 1200 of the catheter tube 1000 can pass through the gap 5000 and into the through-hole 2400 when attaching the fixation device 2000, 2000A, 2000B, 2000C to the catheter tube in situ. The provision of the gap 5000 may facilitate positioning a catheter tube 1000 in the through-hole 2400 in situ without removal of the catheter tube 1000. For instance, the fixation device 2000, 2000A, 2000B, 2000C can be moved, e.g. slid, with respect to the catheter tube 1000, thereby allowing the catheter tube 1000 to pass through the gap 5000 to position it in the through-hole 2400.

In some embodiments, the body 2100 may be configured to be able to deform to an extent sufficient for a user, e.g. a clinician, to manually open the gap 5000 to pass the second portion 1200 of the catheter tube 1000 through the gap 5000. For instance, the clinician can flex the body 2100 to open the gap 5000 and slide the body 2100 into place around the second portion 1200 of the catheter tube 1000. In such embodiments, the gap may be narrower than the width of the catheter tube 1000.

The gap 5000 may separate parts of the body 2100 such that it has a pair of arms 2170. The arms 2170 each have a free end 2172 which is/are distal to the through-hole 2400.

However, in other forms which are not shown, the body 2100 is configured to form a gap but the gap may not be present when the body has no forces acting upon it. For example, the arms 2170 may contact each other or overlap in such a situation.

Fluids may leak or ooze from the exit point S1 and/or a wound proximate the exit point S1. The gap 5000 may be configured to allow fluid to drain out and away from the body 2100 when the body 2100 is orientated in a certain way during use. For instance, when the body 2100 is positioned such that that the gap 5000 faces downwards (e.g. in an inferior direction when the patient is upright), gravity may cause the fluid to drain out through the gap 5000, instead of building up and being retained by the body 2100.

The first opening 2112 allows for the passage of air into and out the through-hole 2400 during use. This may reduce or prevent moisture from accumulating within the through-hole 2400 in use, and improve breathability.

### 5.2.5 Window

According to aspects of the present technology, the body 2100 may be configured to space the sidewall 2130 from the exit point S1. In this way, the body creates a window 4000 to a region of the patient's skin S. The region of the patient's skin S is an area of the skin S which is not in contact with the body 2100.

The body 2100 is configured such that the region of the patient's skin S at least partially surrounds the exit point S1. For instance, the region of the patient's skin S extends in an arc about the exit point S1. As illustrated, the body 2100 may be configured such that the region of the patient's skin S substantially surrounds the exit point S1. For instance, the region of the patient's skin S entirely encircles the exit point S1.

When the fixation device 2000, 2000A, 2000B, 2000C is positioned in use on the patient's skin S, the window 4000 forms an opening, and an access path, that allows the clinician, to visually inspect and/or physically access the region of the patient's skin, the exit point S1 and/or the second portion 1200 of the catheter tube 1000. The clinician can view and/or access the region of the patient's skin S via the second opening 2122. Physical access may be, for example, with the clinician's hands or tools, for example tweezers or the like.

The spacing between the sidewall 2130 and the exit point S1 helps isolate the region of the patient's skin S around the exit point S1 from external conditions or agents. Furthermore, this arrangement may reduce or prevent the region of the skin S around the exit point S1 from being compressed. This may help improve the patient's comfort. It may also improve breathability of the fixation device 2000, 2000A, 2000B, 2000C, e.g. it may facilitate oxygenation of the region of the skin S and/or a wound.

The through-hole 2400 provides an aperture into which the catheter tube 1000 can extend in use, away from the patient's skin S e.g. as is illustrated in FIG. 3.

In the illustrated embodiment, the window 4000 is provided by the through-hole 2400 which extends through the body 2100.

As illustrated, the second portion 1200 of the catheter tube 1000 exits from the patient's skin S at the exit point S1 and extends into the through-hole 2400. The body 2100 is configured such that the second portion 1200 of the catheter tube 1000 extends in use from the body 2100 through the through-hole 2400 to the exit point S1, for example from the channel 2500 through the through-hole 2400 to the exit point. Therefore, the channel 2500 is positioned on the sidewall 2130 such that the catheter tube 1000 extends in use from the body 2100 through the through-hole 2400 to the exit point S1. As is perhaps best illustrated in FIG. 3, at least part of the second portion 1200 is bent through the through-hole 2400 when the catheter tube 1000 is in the bent configuration. However, in other forms not shown, it may be substantially straight, i.e. not bent.

Referring to the Figures, the body 2100 is shaped and dimensioned to provide a gap between a portion of the catheter tube 1000, e.g. the second portion 1200 and the side wall 2130.

In addition, the body 4000 is shaped and dimensioned to permit inspection of a region of the surface of the patient's body which surrounds the point at which the catheter tube 1000 is inserted into or exits from the surface of the patient's body in use, e.g. exit point S1 in a patient's skin S as shown in FIG. 3.

As illustrated, the fixation device 2000, 2000A, 2000B, 2000C comprises the window 2400 provided by the through-hole 2400 extending through the body 2100 and a gap 5000 extending through the body 2100 to the through-hole 2400.

In the illustrated embodiment, the fixation device 2000, 2000A, 2000B, 2000C is configured to substantially surround a region of the patient's skin S. For instance, the body 2100 may surround the exit site S1 such that it subtends an angle of at least 270° around the exit site S1. Preferably, it may surround the exit site S1 such that it subtends an angle of substantially 360° around the exit site S1.

The distance between the edge of the first opening 2112, e.g. a proximal region of the sidewall 2130, and the exit point S1 may be between substantially 10 mm and substantially 30 mm, preferably the distance is substantially 20 mm. The size of the window 4000 is configured to allow the region of the patient's skin S located in the window 4000 to be inspected without moving, modifying, or having to remove the body 2100 from the patient's skin S.

A dressing (not illustrated in the Figures) may be applied on or proximate to the exit site S1, e.g. it may be applied to a wound. Therefore, the window 4000 may facilitate access to the wound to apply the dressing and/or access the dressing and/or wound with the fixation device 2000, 2000A, 2000B in vivo.

The through-hole 2400 and window 4000 can also permit placement of the fixation device 2000, 2000A, 2000B, 2000C around a suture (not shown in the Figures) at a base of the catheter tube 1000 and/or removal of sutures with the fixation device 2000 in vivo. For example, clinicians can apply the fixation device 2000, 2000A, 2000B in addition to sutures when the risk and/or implications of dislodgement of the catheter tube 1000 are higher than normal. In at least some embodiments, the fixation device 2000, 2000A, 2000B is configured to allow access to the wound to facilitate removal of the sutures without disturbing the fixation device 2000, 2000A, 2000B.

The shape and dimensions of the side wall 2130 can vary around its perimeter. For instance, the side wall 2130 comprises at least a first sidewall section 2132 and a second sidewall section 2134. The first sidewall section 2132 is a region of the sidewall 2130 which is distal to the proximal surface 2110 (and the patient's skin S in use), i.e. a distal region of the sidewall 2130. The second sidewall section 2134 is a region of the sidewall 2130 which is proximal to the proximal surface (and the patient's skin S in use), i.e. a proximal region of the sidewall 2130.

The shape of the first sidewall section 2132 may be different from the second sidewall section 2134. For instance, referring to FIG. 11, the first sidewall section 2132 is arcuate, and the second sidewall section 2134 is substantially straight. However, in other forms, the shape of the first sidewall section may be similar to or the same as the second sidewall section, e.g. both sections are substantially arcuate or straight.

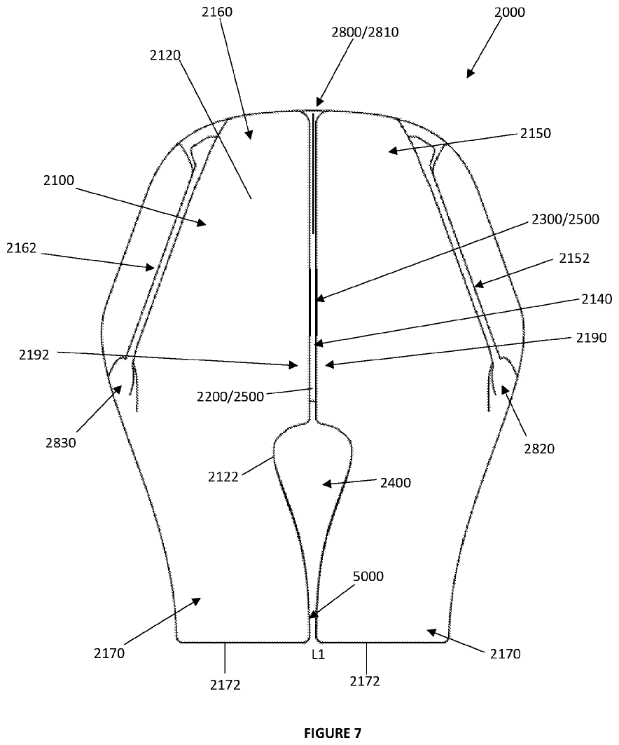

As illustrated in FIGS. 6 and 7, the first opening 2112 and second opening 2122 are each substantially teardrop shaped. In alternate forms, the first opening and/or the second opening 2122 may be any other suitable shape, e.g. circular or oval-shaped.

### 5.2.6 Overhang

According to an aspect of the present technology, the fixation device 2000, 2000A, 2000B, 2000C comprises an overhang 2700. As illustrated, the sidewall 2130 may be configured to form the overhang 2700.

The shape of the side wall 2130 may provide the overhang 2700. For instance, in at least a region of the sidewall 2130, the edge of the first opening 2112 is offset from the edge of the second opening 2122. In other words, there is at least a region of the sidewall 2130 which is configured to protrude into the through-hole 2400 to a greater extent than an adjacent region of the sidewall 2130.

As illustrated, an end of the channel 2500 may be provided in the overhang 2700. For example, the first channel opening 2530 may be positioned on the sidewall 2130 in, or just above, the overhang 2700.

As illustrated, the sidewall 2130 is structured to create the overhang 2700 which provides the space between the second portion 1200 of the catheter tube 1000 and the sidewall 2130.

As is perhaps best illustrated in FIGS. 3, 24 and 48, the overhang 2700 spaces the second portion 1200 of the catheter tube 1000 from the sidewall 2130. This creates a substantial space between the second portion 1200 at the exit point S1 and the body 2100 where it exits from the channel 2500, for example. Therefore, the catheter tube 1000 does not contact the body 2100 at the exit point S1. This reduces or limits the creation of local pressure on the skin S proximate the exit point S1 which can occur when a catheter tube contacts or is proximate a body of a fixation device, i.e. it lies against or is close to the body at its exit point.

In addition, the overhang 2700 helps create the window 4000 to the region of the patient's skin S that surrounds the exit point S1. It does this by spacing the second portion 1200 of the catheter tube 1000 from the sidewall 2130 located beneath the overhang 2700.

It can also be seen from the Figures, and more particularly FIG. 40, that in the preferred form the first opening 2112 is relatively larger than the second opening 2122. The first opening 2112 may define a first perimeter, and the second opening 2122 may define a second perimeter, wherein the first perimeter is larger than the second perimeter. This may contribute to creating the overhang 2700.

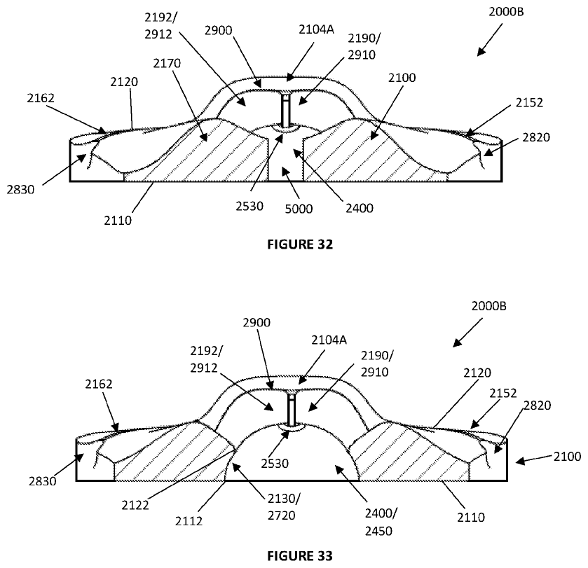

The shape and/or dimensions of the side wall 2130 may be varied around the periphery of the through-hole 2400. The variation in shape around the side wall 2130 may define at least a first overhang region 2710 and a second overhang region 2720. FIGS. 24 and 35 show an example of the first overhang region 2710. FIG. 33 shows an example of the second overhang region 2720. The first overhang region 2710 has a different amount of overhang compared to the second overhang region 2720. As illustrated, the first overhang region 2710 has a larger overhang compared to the second overhang region 2720. The variation in dimensions, e.g. distance between the edge of the first opening 2112 and the edge of the second opening 2122 (referred to also as “height”), around the sidewall 2130 create sections of the sidewall 2130 which different heights. For instance, the height(s) of the sidewall 2130 in the first overhang region 2710 may be less than the height of the sidewall 2130 in the second overhang region 2720, as illustrated in FIGS. 24 and 33. Furthermore, FIG. 32 shows that some sections of the sidewall 2130 may not have an overhang, however, the height may be different to other sections of the sidewall 2130.

The first overhang region 2710 and the second overhang region 2720 may be structured to provide separation from the second sidewall section 2134, and the second portion 1100 of the catheter tube 1000 in use. The separation may be useful to assisting the catheter tube 1000 to be placed in a bent configuration and direct the catheter tube 1000 into the channel 2500. The second overhang region 2720 may also surround a part of the region of the patient's skin S in the window 4000.

The overhang 2700, e.g. the first overhang region 2710, may be configured to limit or prevent the second portion 1200 of the catheter tube 1000 from being pushed too far into the window 4000 when positioning the fixation device 2000, 2000A, 2000B, 2000C around a catheter tube in situ. This helps position the exit point S1 in the desired location within the window 4000, e.g. at a mid-point between opposing sides of the body 2100, and spaced apart from the sidewall 2130 below a proximal edge of the section portion 1200. For instance, when the second portion 1200 contacts the overhang 2700, this indicates to the clinician that the second portion 1200 is correctly placed in the window 4000 and catheter tube 1000 can be inserted into the channel 2500. This may help stop the clinician from pushing the catheter tube 1000 too far into the window 4000 so it kinks or buckles.

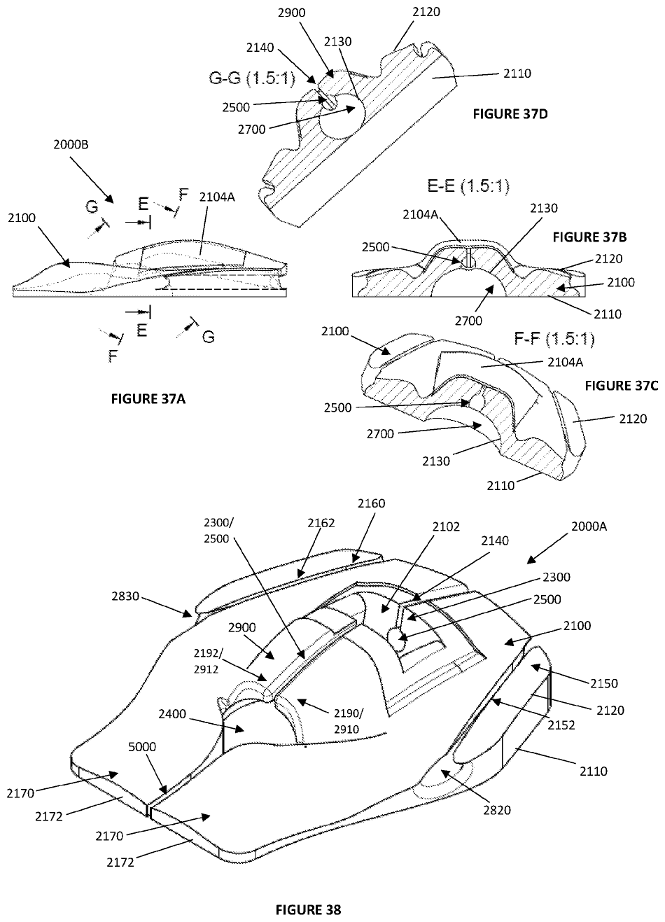

FIGS. 37A-37D also shows the variation in the shape of the sidewall 2130 around the periphery of the window 2400 which defines different overhangs.

### 5.2.7 Chamber

According to aspects of the current technology, the fixation device 2000, 2000A, 2000B, 2000C may comprise a chamber 2450, e.g. the body 2100 may be shaped in such a way that a chamber 2450 is defined. In some forms, the sidewall 2130 may be shaped and/or dimensioned to create a chamber 2450 around the exit point S1 in use.

The chamber 2450 may be concave, for example substantially dome-shaped, as is perhaps best illustrated in FIGS. 33, 55, 37-B to 37-D, 48, and FIGS. 52 and 53. However, it is appreciated that in other forms, the chamber 2450 may have other shapes.

It is to be appreciated that reference to “substantially dome-shaped” in the case of a cavity in the underside of the fixation device refers to the shape of the negative space created inside the cavity. That space may be “substantially” dome-shaped in that options for the shape include a partial dome-shape. Therefore, the chamber 2450 does not necessarily form a complete dome-shape or complete dome, but forms at least a partial dome-shape or partial dome. The dome-shaped chamber 2450 may also be referred to as a “dome-like structure”. Furthermore, reference to a “chamber” may be understood to refer to a chamber that at least partially encloses a space or volume, therefore, it does not necessarily completely enclose the space or volume.

In use, the sidewall 2130 extends away from the patient's skin S, and is configured to converge to substantially enclose a volume or space which substantially surrounds the exit point S1, and create the chamber 2450. The sidewall 2130 forms the wall(s) of the chamber 2450. Therefore, the sidewall 2130 is shaped and dimensioned to create the chamber 2450.

The chamber 2450 encapsulates, or (in use) borders, at least part of the region of the patient's skin S. This forms a shield-like space which substantially surrounds and substantially encloses the region of the patient's skin S and the second portion 1200 of the catheter tube 1000, whilst providing visibility of the region of the patient's skin S and the second portion 1200 in combination with the window 4000. The sidewall 2130 extends away from the skin S in use, and over the second portion 1200. The sidewall 2130 is spaced from the second portion 1200 apart from where the catheter tube 1000 enters the channel 2500.

The chamber 2450 formed in the body 2100 is configured to allow for forces that are applied to the body 2100 in use to be distributed away from the region of the patient's skin S and the exit point S1. This may help protect the catheter tube 1000, a wound in which the exit point S1 is located, and a dressing which can be positioned on or proximate the exit point S1. For example, a downward force exerted on body 2100 may be transmitted down through the body around the chamber 2450 so as to transmit the force onto the skin at points distanced from the exit point.

In the embodiments of the technology illustrated in FIGS. 22 to 55, the fixation device 2000A, 2000B, 2000C comprises a cover 2900 configured to cover at least a section of the window 4000. The cover 2900 extends across at least a portion of the window 4000. The body 2100 may be open in a section of the window 4000 that is not covered by the cover 2900. The second opening 2122 is not covered in that section and provides some visibility and access into the window 4000 without having to move the cover 2900.

In the illustrated forms, the cover 2900 extends from the sidewall 2130. However, in other forms which are not shown, the cover 2900 can extend from one or more other parts of the body 2100.

As illustrated, the sidewall 2130 and cover 2900 may together create the dome-shaped chamber 2450.

The cover 2900 may be movable. The cover 2900 may be resiliently biased to a position in which it overlays the window, e.g. it is formed form a resilient material. When the cover 2900 is moved it increases the size of the second opening 2122, and allows more visibility and access into the window 4000.

As illustrated, in some forms of the technology, the sidewall 2130 is configured to form the cover 2900, e.g. the side wall 2130 may be configured to form one or more cover flaps 2910, 2912, and which extend over at least a portion of the window 4000.

In the illustrated form, the one or more cover flaps 2910, 2912 each extend from a side of the body 2100 towards the other side of the body 2100. As illustrated, the cover flap 2910 extends from a first side of the body 2100, and the cover flap 2912 extends from a second side of the body 2100 which is opposite the first side. The cover flaps 2910, 2912 extend toward each other such that an edge of cover flap 2910 is substantially parallel to, and opposite, an edge of cover flap 2912. The edges of the respective cover flaps 2910, 2912 are spaced apart from each other to create part of the slit 2140. However, in other forms not shown, one cover flap may overlap or contact another cover flap.

The cover 2900 may be configured to retain the first portion 1100 of the catheter tube 1000 in the channel 2500. For instance, the cover flaps 2910, 2912 may form part of the channel 2500. A proximal surface, i.e. underside, of each of the cover flaps 2910, 2912 may provide a portion of the channel 2500. The portion of the channel 2500 may be resiliently biased against a surface of the catheter tube 1000 in that region, as is perhaps best shown in FIGS. 24 and 25. Therefore, the cover 2900 may act as the retainer 2300.

Therefore, the cover 2900 may help retain the first portion 1100 of the catheter tube 1000 in the channel 2600 and may additionally, or alternatively, allow access to or visibility through the window 4000 by moving the cover 2900, e.g. peeling back the cover flaps 2910, 2912.

The cover flaps 2910, 2912 may be substantially thinner and more flexible than adjacent portions of the body 2100. This allows them to be moved, e.g. peeled apart, more easily to facilitate access and/or inspection through the window 4000.

In the illustrated forms, the first and second sides 2190, 2192 in the region of the first channel opening 2530 may be integrally formed with the cover 2900 such that the first and second sides 2190, 2192 extend away from the channel 2500 across at least a portion of the window to form the cover 2900. The slit 2140 may extend to a distal end of the cover 2900. However, in other forms not shown in the figures, it is to be appreciated that the cover may not be integrally formed with the sidewall and may be provided by a separate component.

Referring again now to FIGS. 22 to 25, the cover 2900 extends above at least part of the second portion 1200 of the catheter tube 1000 that extends through the through-hole 2400. As illustrated, the cover 2900 extends above the first portion 1100 to substantially cover it. This may help isolate the exit point S1 and second portion 1200 so it is protected. This may reduce or limit the possibility of the second portion 1200 being snagged or inadvertently tugged during use.

FIGS. 24 and 25 illustrate the overhang 2700 of the sidewall 2130 forms the cover 2900. As is shown, the retainer 2300 overhangs the bearing surface 2200, e.g. a distal channel surface 2520 overhangs a proximal channel surface 2510. This helps retain the catheter tube 1000 in the bent configuration. The distal channel surface 2520 may form part of the cover 2900.

### 5.2.8 Fixation Device Profile

In certain forms, the fixation device 2000, 2000A, 2000B, 2000C may have a relatively low profile. The distal surface 2120 may be substantially smooth. Therefore, in some forms, the fixation device 2000, 2000A, 2000B, 2000C has a relatively smooth profile. The distal surface 2120 may be contoured and may have no exposed edges. This may reduce or prevent the fixation device 2000, 2000A, 2000B from snagging or catching on objects. The proximal surface 2110 may be substantially flat, which allows it to come into contact with the surface of a patient's skin S.

In certain forms, the body 2100 may be configured such that, when viewed from a side in a direction substantially parallel to the proximal surface 2110, the distal surface 2120 has a substantially convex-shaped envelope. Therefore, when viewed from the side, the body 2100 is substantially convex-shaped.

Referring to FIG. 9, height h1 is the distance between a point on the proximal surface 2110 and the highest (distal most) point of the bearing surface 2200. Height h1 may contribute to the low profile of the fixation device 2000, 2000A, 2000B. For instance, the height h1 is chosen to allow the catheter tube 1000 to be held in a bent configuration having a curved region which curves without kinking about a radius which is equal to or less than the bend radius of the catheter tube 1000.

In at least some embodiments, the fixation device 2000, 2000A, 2000B, 2000C is configured to distribute forces, applied to the distal most regions, across the footprint of the fixation device 2000, 2000A, 2000B, 2000C to alleviate deformation of the catheter tube 1000 by forces that can be exerted during treatment. For example, the shape of the fixation device 2000, 2000A, 2000B, 2000C shown in FIG. 6 can, in some instances, distribute forces outwardly away from the catheter tube (not shown in FIG. 6) when a patient is lying on the fixation device 2000, 2000A, 2000B, 2000C. In some embodiments, the fixation device 2000, 2000A, 2000B, 2000C is configured to deform around the channel 2500 when force is applied to the distal most regions to alleviate displacement of the catheter tube 1000.

Referring now to FIGS. 2 and 3, the catheter tube 1000 is retained inferior to the distal most point of the distal surface 2120 because the channel 2500 is structured to be below the distal most point.

Height h2 is the highest point of the body 2100 from the patient's skin S. Height h3 is the highest point on the catheter tube 1000 in its bent configuration. Height h2 is larger than height h3. The catheter tube 1000 is therefore entirely below the distal most point of body 2100. When the patient lies on the body 2100, this arrangement may help reduce or prevent compression of the catheter tube 1000, which may occlude it. The distal surface of the first portion 1100 of the catheter tube 1000 is positioned away from the distal surface 2120 which receives the forces.

The catheter tube 1000 is retained inferior to the second opening 2122 as at least a portion of the body 2100 is superior to the first channel opening 2130. This may facilitate protecting the catheter tube 1000, therefore reducing or preventing it being crushed in use.

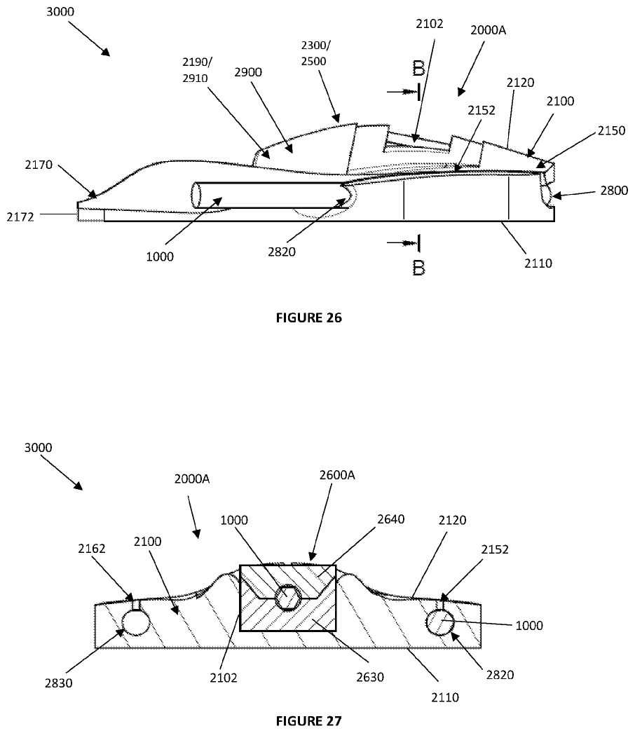

The height of the sidewall 2130 may vary around the perimeter of the through-hole 2400. As illustrated, the height of the sidewall 2130 increases towards the bearing surface 2200, e.g. from the pair of arms 2170 towards the channel 2500. The height of the sidewall 2130 may be varied to match the variation in height of the body 2100. The height of the body 2100 increases from the free ends 2172 towards the channel 2500 up to the highest point on the body 2100, after which the height of the body 2100 decreases. This is perhaps best seen in FIGS. 13 and 26. This creates the substantially convex-shaped envelope. This shape may facilitate creating a low-profile fixation device 2000, 2000A, 2000B. It is to be envisaged that in other embodiments, the body 2100 may be formed in any other suitable shape.

When viewing the body 2100 from an end, for example, as shown FIG. 23, the height of the body 2100 increases from a first side towards the channel 2500 up to the highest point on the body 2100, after which the height of the body 2100 decreases towards the other side.

Referring to FIG. 7, the width of the body 2100 is the distance between the sides of the body 2100, and the length is the distance between the ends of the body 2100. The width may be less than the length of the body 2100. This may create a substantially elongate body 2100. The height of body 2100 (which is preferably varied), e.g. height h2, may be less than each of the width and the length. This helps create the low-profile fixation device 2000, 2000A 2000B, 2000C.

The width of the body 2100 may be significantly larger than the diameter of the catheter tube 1000. Additionally, or alternatively, the width of the body 2100 may be a significant portion of the length of the body 2100. Therefore, the body 2100 is generally elongate but has a substantially wide footprint. This may improve attachment of the body 2100 to the patient's skin S, e.g. allow for better adhesion to the patient's skin S.

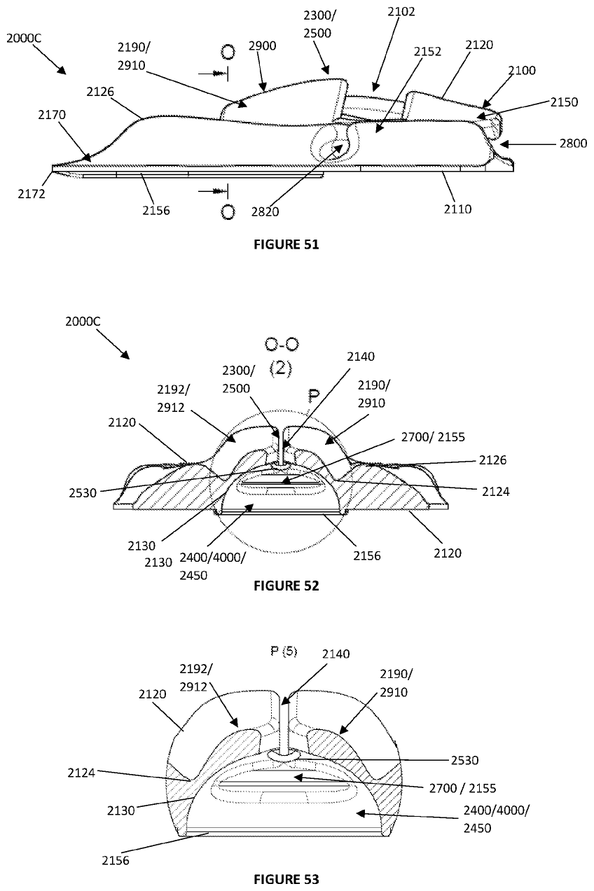

As is perhaps best shown in FIGS. 51 to 53, in some embodiments, the body 2100 comprises one or more thinner regions or recesses 2124 and/or one or more thicker regions or ridges 2126.

For instance, body 2100 has recesses where weight or thickness of the body 2100 can be reduced without impacting the performance of the body 2100, e.g. impact the ability to distribute forces and/or flex to contour to the patient's skin S. This may reduce the weight of the body 2100. This may also allow less material to be used to manufacture the body 2100. The recess(es) 2124 and/or ridge(s) 2126 may help support loading on top of the body 2100 without transmitting forces directly to the catheter tube 1000 or region of the patient's skin in the window 4000. For example, recesses 2124 may be provided in the distal surface in a region above and adjacent the location of chamber 2450.

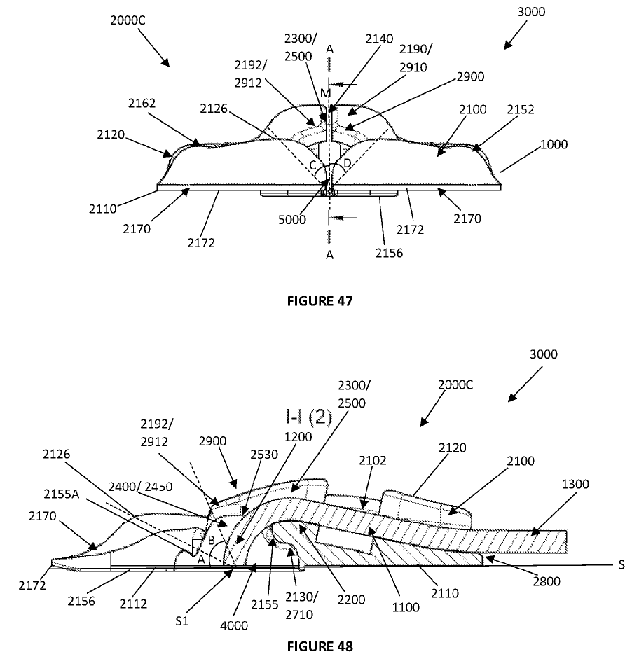

As is perhaps best shown in FIGS. 47 and 48, the distal surface 2120 may be shaped to form ridges 2126 where the body is thicker than in adjacent regions. For instance, each arm 2170 may comprise a ridge 2126. The distal surface 2120 of each arm 2170 may slope upwards from a side of the body 2100 and from the free end 2172 to a highest point of the ridge 2126, and thereafter the distal surface 2120 may slope downwards to the sidewall 2130. The ridges 2126 may create a barrier or shield around the through-hole 2400. This may help further protect the skin S located in the window 4000.

Referring to FIG. 48, when viewed in cross-section from a side (i.e. from a direction perpendicular to the direction of the length of the channel 2500) in a direction substantially parallel to the proximal surface 2110, the entrance of the window 4000 (i.e. the upper opening in the through-hole 2400) subtends an angle at the exit point S1. The angle subtended by the entrance of the window 4000 at exit point S1 may be between approximately 20° and approximately 50°. Put another way, this angle may be considered as the angle between a line extending from exit point S1 to the bottom region of the window 4000 and a line extending from exit point S1 to a top region of the window 4000, when viewed in the stated cross-section (as per FIG. 48). The top region of the window 4000 in this context may generally be defined by an end region of the cover 2900, e.g. the cover flaps 2910, 2912, discussed above. This angle is labelled as angle B in FIG. 48.

Also marked in FIG. 48 is angle A, which in this form is the angle between a plane defined by the proximal surface of the fixation device (or equivalently, the plane of the patient's skin S when the device is in use) and a line extending from exit point S1 to a bottom region of the window 4000. The bottom region of the window 4000 may generally be defined by a top region of the ridges 2126 described above. Angle A may be between approximately 10° and approximately 30° in certain exemplary forms.

Referring to FIG. 47, when viewed in cross-section from an end (i.e. from a direction parallel to the direction of the length of the channel 2500) in a direction substantially parallel to the proximal surface 2110, the entrance of the window 4000 (i.e. the upper opening in the through-hole 2400) subtends an angle at the exit point S1. The half-angle subtended by the window 4000 at the exit point S1 (i.e. angle C and/or angle D in FIG. 47) may be between approximately 40° and approximately 120°. Angle C is the angle between mid-line M and a line extending from exit point S1 to a first side region of the window 4000. Angle D is the angle between mid-line M and a line extending from the exit point S1 to a second side region of the window 4000. The first and second side regions of the window 4000 may generally be defined by a side region of each of the ridges 2126.

If angles B, C or D are too small, for example smaller than the respective lower limit indicated above, then it may become overly difficult for a dressing to be applied/removed through the window 4000, and visibility and/or access may be decreased. Similarly, this may also occur if angle A is decreased without also increasing angle B. If angles B, C or D are too great, for example larger than the respective upper limit indicated above, then a dressing and the second portion 1200 of the catheter tube 1000 may become more exposed, and it may become overly easy for the dressing to be inadvertently removed.

### 5.2.9 Lead-In

In some forms of the technology, the fixation device 2000 comprises a lead-in 2915 to the body 2100. For example, the lead-in 2915 may be to the channel 2500.

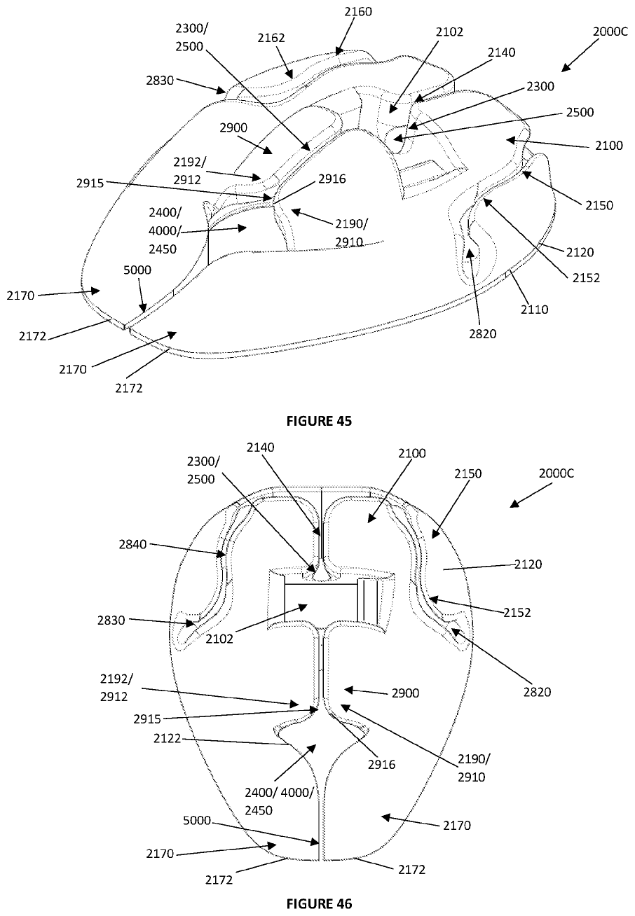

As is perhaps best illustrated in FIGS. 45 and 46, the body 2100 may be configured to form the lead-in 2915 to the slit 2140 to facilitate insertion of the catheter tube 1000 into the channel 2500 in use. The sidewall 2130 and/or cover 2900 may be structured to create the lead-in 2915. For instance, as illustrated, an end of each cover flap 2910, 2912 comprises a rounded edge 2916 proximate the slit 2140 which together provide the lead-in 2915 when the cover flaps 2910, 2912 are in the neutral configuration.

The lead-in 2915 may help ensure correct alignment between the body 2100 and the catheter tube 1000 when the fixation device 2000, 2000A, 2000B, 2000C is positioned around a catheter in situ and facilitates insertion of the first portion 1100 into the channel 2500.

In other forms not shown, other parts of the body 2100 may be configured to form a lead-in to the gap 5000. For instance, an end of each arm 2170 comprises a rounded edge proximate the gap 5000 which together provide a lead-in.

As is perhaps best shown in FIGS. 50 and 54, in certain forms, the overhang 2700 as described above, when viewed in cross-section from the top in a direction substantially perpendicular to the proximal surface 2110, may have a substantially concave shape. This may provide a lead-in to the channel 2500 to help locate the catheter tube centrally in the fixation device when the fixation device is slid onto the catheter tube in situ.

### 5.2.10 Exit Pathways/Channels

In embodiments of the technology, the fixation device 2000, 2000A, 2000B, 2000C comprises one or more exit pathways. In preferred forms, the exit pathway(s) is/are provided by one or more channels 2800 formed in the body 2100.

In the illustrated embodiment, the fixation device 2000, 2000A, 2000B comprises a first exit pathway configured to direct the catheter tube 1000 away from the body 2100 in a first direction. The body 2100 comprises a first channel 2810 which may, in use, receive a first portion 1100 of the catheter tube 1000. The channel 2500 provides the first channel 2810. The first channel 2810 is configured such that the catheter tube 1000 extends away from the body 2100 in a first direction when the first portion 1100 of the catheter tube 1000 is positioned in the first channel 2810.

As shown in FIG. 1, the first direction may be substantially straight. The channel 2500 forms the first channel 2810. The third region 2230 which has been described above, extends from the second region 2220 to the second channel opening 2540 to define the first channel 2810. FIG. 3 shows the first direction is substantially parallel to the patient's skin surface S.

In the illustrated embodiment, the fixation device 2000, 2000A, 2000B, 2000C comprises one or more channel wall sections 2182, 2184 in the region of the first exit pathway 2810. The channel wall section(s) 2182, 2184 are located adjacent the second channel opening 2540. In some forms, the channel wall section(s) 2182, 2184 are curved, as is perhaps best shown in FIG. 50, and may be formed on either side of the second channel opening 2540. The channel wall sections 2182, 2184 are configured to support the catheter tube 1000 and permit further bending of the catheter tube 1000 along a plane which is substantially parallel to the patient's skin, without causing it to kink or occlude. This may allow for the direction that the catheter tube 1000 extends from the body 2100 to be changed. For example, channel wall sections 2182, 2184 are shaped to hold the catheter tube 1000 in a bent configuration with an angle of up to substantially 45° towards either side of the first channel 2810. This may provide clinicians with more freedom to position and/or reposition the catheter tube 1000.

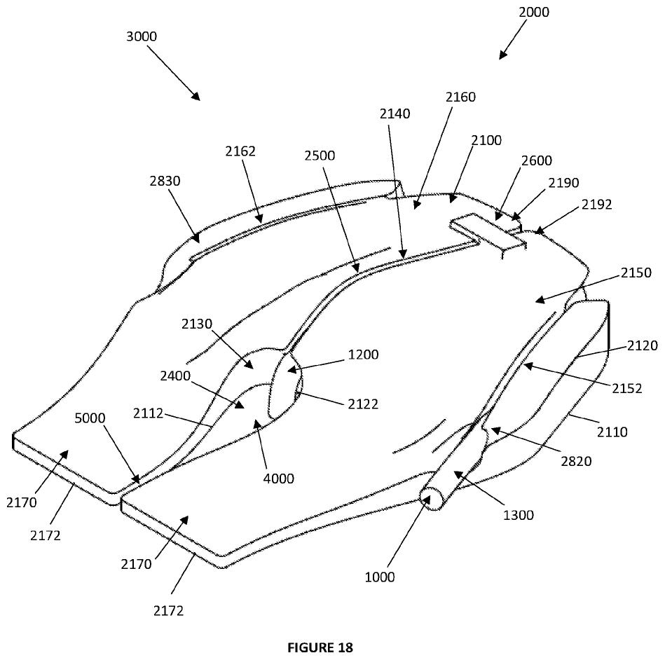

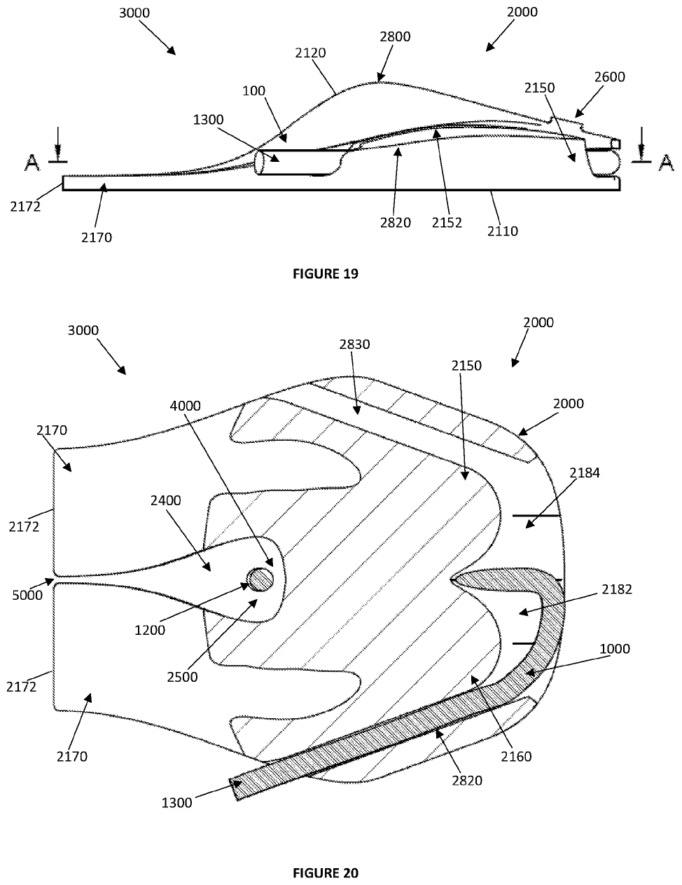

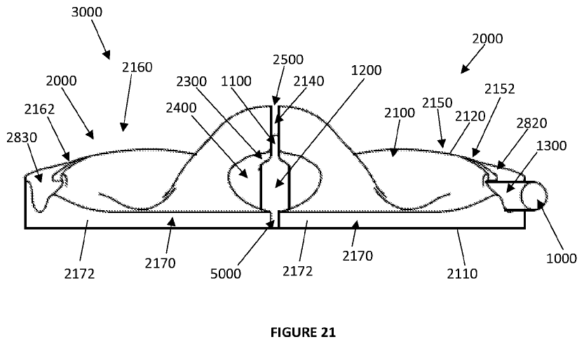

In the illustrated embodiment of FIGS. 18 to 21, the fixation device 2000, 2000A, 2000B comprises a second exit pathway configured to direct the catheter tube 1000 away from the body 2100 in a second direction. The body 2100 further comprises a second channel 2820 formed in the distal surface 2120 which, in use, may receive the first portion 1100 of the catheter tube 1000 as an alternative to the first portion 1100 of the catheter tube 1000 being received in the first channel 2810. The second channel 2820 is configured such that the catheter tube 1000 extends away from the body 2100 in a second direction when the first portion 1100 of the catheter tube 1000 is positioned in the second channel 2820.

FIG. 20 shows that the second channel 2820 is configured to bend the catheter tube 1000 around a section 2150 of the body 2100 without occluding it. The second channel 2820 comprises a slit 2152, similar in structure to slit 2140, and configured to facilitate insertion/removal of a portion of the catheter tube 1000 which is bent around section 2150.

The first direction may be substantially different from the second direction. The angle between the first direction and the second direction may be between substantially 45° and substantially 180°, preferably between substantially 90° and substantially 180°, and more preferably between substantially 120° and substantially 150°. These aspects of the technology may facilitate matching the direction that the catheter tube 1000 exits from the fixation device 2000, 2000A, 2000B to the patient's orientation and/or position, e.g. supine or prone position. By enabling the direction of the catheter tube 1000 to be changed the catheter tube 100 can be repositioned on the body 2100 to reduce or limit kinking and/or snagging events from occurring. In addition, the first channel 2810 and second channel 2820 are configured to substantially surround and enclose the catheter tube 1000 to protect it and reduce or limit snagging events.

The first channel 2810 may be the full path that the catheter tube 1000 is positioned in when in a first configuration. The second channel 2820 may be another full path that the catheter tube 1000 is positioned in when in a second configuration. The second channel 2820 may be different in part to the first channel 2810, but the channels 2810, 2820 may share one or more channel portions.

Therefore, the body 2100 may comprise a channel portion which is common to the first channel 2810 and to the second channel 2820. As illustrated, this common channel portion is the first channel 2810. For instance, when the catheter tube 1000 is in the second configuration, part of the catheter tube 1000 extends through channel 2810 and another part extends through channel 2820.

In other forms not shown, the channels may be separate channels without shared channel portions.

As illustrated in the Figures, the fixation device 2000, 2000A, 2000B comprises a third exit pathway configured to direct a catheter tube away from the body 2100 in a third direction (not shown in the Figures). The body 2100 further comprises a third channel 2830 formed in the distal surface 2120 which, in use, may receive the first portion 1100 of the catheter tube 1000 as an alternative to the first portion 1100 of the catheter tube 1000 being received in the first channel 2810 or the second channel 2820. The third channel 2830 is configured such that the catheter tube 1000 extends away from the body 2100 in a third direction when the first portion 1100 of the catheter tube 1000 is positioned in the third channel 2830.

The third channel 2830 is configured to bend the catheter tube around a section 2160 of the body 2100 without occluding it. The third channel 2830 comprises a slit 2162 which may be the same as slit 2152, for insertion/removal of a portion of the catheter tube which is bent around section 2160. The third direction is different to the first direction and/or the second direction.

At least one of the channels of the second channel 2820 and the third channel 2830 is dimensioned to apply a biasing or restricting force to reduce or eliminate buckling of a portion of the catheter tube disposed in the channel(s) 2820, 2830.

As is perhaps best shown in FIGS. 46 and 50, the body 2100 may be configured such that, when viewed in cross-section from the top in a direction substantially perpendicular to the proximal surface 2110, the second channel 2820 and/or the third channel 2830 comprise one or more curved regions 2840 which bend the third portion 1300 of the catheter tube 1000. The curved regions 2840 may be substantially S-shaped. This shape helps retain the catheter tube 1000 in the second channel 2820 or the third channel 2830 when the third portion 1300 is positioned therein.

### 5.2.11 Attachment to a Patient's Skin

The fixation device 2000, 2000A, 2000B is configured to be attached to the surface of the patient's body. The fixation device 2000, 2000A, 2000B may comprise an attachment component to facilitate attachment of the body 2100 to the surface of the patient's body.

In embodiments of the technology not shown in the Figures, the attachment component may comprise a layer of adhesive provided to the proximal surface 2110 for attachment to the patient's skin. This may facilitate suture-free attachment of the fixation device 2000, 2000A, 2000B to the patient's body. In embodiments which are also not illustrated, the attachment component may comprise a structure or component configured to receive at least one stitch or suture. The structure or component may be formed as a part of the body 2100, or permanently or releasably attached to the body 2100 to facilitate attachment to the patient's body.

### 5.2.12 Dressing Retention

In certain forms, including the embodiment of the technology illustrated in FIGS. 44 to 55, the fixation device 2000, 2000A, 2000B, 2000C is configured to retain a dressing (not shown) in position on the patient's skin S during use. The dressing may be applied to a region of the patient's skin S around the exit point S1. The dressing may be a non-adhesive dressing. Therefore, the fixation device 2000, 2000A, 2000B, 2000C may retain the dressing without using a dressing adhesive or other dressing fastener, e.g. tape or a clip. Removing the need to use an adhesive or fastener can help reduce or prevent irritation or the creation of additional pressure on the patient's skin. The dressing type can be selected and/or changed depending on the leakage occurring from a wound at the exit point S1. Therefore, the fixation device 2000, 2000A, 2000B, 2000C is configured to allow for the dressing type to be changed more easily.

Dressing retention is provided by the patient's skin S and the sidewall 2130, in combination. The dressing may be retained between the patient's skin S and the sidewall 2130. Features of the sidewall 2130 as described herein, e.g. the overhang 2700, may help retain the dressing in position. Furthermore, the chamber 2450 may help retain the dressing when it is positioned within the chamber 2450. The chamber 2450 at least partially surrounds and encloses the dressing to retain it. The shape of the chamber 2450, e.g. dome-shaped, may facilitate retention of the dressing. The dressing may be packed, stuffed or tucked into the chamber 2450. The chamber 2450 allows the dressing to be packed inside it under pressure. This may enable absorption of any leakage or oozing from the exit point S1. The wall(s) of the chamber 2450 provide a surface which pushes against the dressing in use.

The size of the through-hole 2400 may have dimensions so that it can accommodate a standard dressing size. The sidewall 2130 is spaced from the exit point S1 such that the dressing can easily be applied around the exit point S1. The catheter tube 1000 may help retain the dressing because it provides another surface which the dressing can be positioned between, e.g. between the surface of the catheter tube 1000 and the sidewall 2130.

The cover 2900, e.g. the cover flaps 2910, 2912, may be moved, e.g. flexed away from the body 2100 to allow for a dressing to be applied or inspected, and thereafter the cover flaps 2910, 2912 may resiliently return to a neutral configuration such that they retain the dressing in place.

The fixation device 2000, 2000A, 2000B, 2000C may comprise a dressing retainer configured to retain the dressing in position relative to the patient's skin S in use. For instance, the body 2100 may comprise one or more protrusions 2155 configured to grip the dressing which is positioned on or proximate the exit point S. The protrusions 2155 may be formed on at least one of the sidewall 2130 and the proximal surface of the cover flaps 2910, 2912.

As mentioned, the sidewall 2130 can be configured to retain the dressing, so it may act as the dressing retainer. For example, as is perhaps best shown in FIG. 48, the overhang 2700 formed by the sidewall 2130 may be sufficiently spaced apart from a plane of the proximal surface of the body to allow a dressing to be tucked under the overhang 2700. In this embodiment, the first overhang region 2710 is different from those in the other embodiments described and illustrated herein. For example, the first overhang region 2710 has a protrusion 2155 which is thicker and protrudes further into the through-hole 2400 when compared to the other embodiments of the present technology which are disclosed herein. The protrusion 2155 may extend around part of the periphery of the window 4000, e.g. it extends in an arc about the exit point S1 in use. The dressing can be tucked under a proximal surface of the protrusion 2155 to grip the dressing.

Another protrusion 2155A may be formed on the sidewall 2130, and is shown in FIG. 48. The protrusion 2155A may be a hook-shaped ridge which extends or points towards the patient's skin in use, for example. The protrusion 2155A may be provided in a different section of the sidewall 2130 to the protrusion 2155, for example on an opposite side of the through-hole 2400 to protrusion 2155. These protrusions 2155, 2155A may individually or together form a ledge under which the dressing can be positioned to help retain it.

In other examples not shown, at least one of the cover flaps 2910, 2912 comprises one or more protrusions (not shown). The protrusion(s) may be formed on the proximal surface, i.e. underside, of the cover flaps 2910, 2912. For instance, the protrusion(s) may extend from an end region of each of the cover flaps 2910, 2912 towards the patient's skin S in use.

In another example not shown, the protrusion(s) may be in the form of one or more hooks formed on at least one of the proximal surface of the cover flaps 2910, 2912 and the sidewall 2130.

In yet another example not shown, at least one of the sidewall 2130 and the proximal surface of the cover flaps 2910, 2912 may comprise a textured surface which can grip the dressing.

The embodiment shown in FIGS. 44 to 55 comprises a protrusion 2156 formed on the proximal surface 2110. The protrusion 2156 may extend around the edge of the first opening 2112.

The protrusion 2156 may seal with the patient's skin S in use to reduce or prevent fluid from draining or leaking between the proximal surface 2110 and the patient's skin S. The protrusion 2156 may isolate in use any fluids in the through-hole 2400. Drainage or leakage can negatively impact adhesion of the body 21 to the skin S in use, for example, the life-span of the adhesive layer used to attach the body 2100 may be substantially reduced. In addition, this arrangement helps protect the skin S positioned under the proximal surface 2110 from fluid ingress during use.

The protrusion 2156 is positioned on or proximate the edge of the first opening 2112. The protrusion 2156 may be positioned proximate the edge of the first opening 2112 to ensure that the protrusion 2156 does not compromise in use the circulation in the region of the skin around the exit point S1, e.g. where a wound is located.

In some forms, the protrusion 2156 may grip a dressing which is positioned in use between the proximal surface 2110 and the patient's skin S.

### 5.2.13 Construction

In embodiments of the technology, the components of the fixation device 2000, 2000A, 2000B described herein are formed as a single unit. The fixation device 2000, 2000A, 2000B, as illustrated, may be integrally formed, e.g. the body 2100 may be injection moulded or the components of the fixation device 2000, 2000A, 2000B described above may be separately formed and co-moulded to each other.

In an embodiment, the body 2100 or parts of it may be constructed from a relatively resilient material, e.g. one or more of a suitable polymer such as silicone or polyurethane, a synthetic rubber, and other medical grade material(s) with suitable resilience and/or flexibility. In some forms, the body 2100 may comprise one or more hollow sections.

The structure of the fixation device 2000, 2000A, 2000B as described herein, and the material properties described above may facilitate flex, movement, and/or compression of the body 2100 or parts of it when a force is applied to it. For instance, construction of the fixation device 2000, 2000A, 2000B may allow it to conform to the contours of the patient's skin S. This may allow the fixation device 2000, 2000A, 2000B to be used on different parts of the patient's body e.g. flat or substantially curved parts. In addition, this construction may help distribute the forces applied to the fixation device 2000, 2000A, 2000B when the fixation device 2000, 2000A, 2000B is compressed, e.g. the patient lies on it. This may improve patient comfort.

In another embodiment, the body 2100 or parts of it may be constructed from a relatively rigid material. This may reduce/prevent flex, movement, and/or compression of the body 2100 or parts of it when a force is applied to it.

Unless the context clearly requires otherwise, throughout the description and the claims, the words “include”, “including”, “comprise”, “comprising” and the like, are to be construed in an inclusive sense as opposed to an exclusive or exhaustive sense, that is to say, in the sense of “including, but not limited to”.

Reference to any prior art in this specification is not, and should not be taken as, an acknowledgement or any form of suggestion that that prior art forms part of the common general knowledge in the field of endeavour in any country in the world.

Aspects of the present technology may also be said broadly to consist in the parts, elements and features referred to or indicated in the specification of the application, individually or collectively, in any or all combinations of two or more of said parts, elements or features. Where in the foregoing description reference has been made to integers or components having known equivalents thereof, those integers are herein incorporated as if individually set forth.

It should be noted that various changes and modifications to the presently preferred embodiments described herein will be apparent to those skilled in the art. Such changes and modifications may be made without departing from the spirit and scope of the invention and without diminishing its attendant advantages. It is therefore intended that such changes and modifications be included within the present technology.

1. A fixation device to secure a catheter tube in a bent configuration relative to a surface of a patient's skin, wherein the fixation device comprises:
a body comprising:
a proximal surface which, in use, faces towards the surface of the patient's skin;
a distal surface which, in use, faces away from the surface of the patient's skin;
a first opening in the proximal surface;
a second opening in the distal surface;
a sidewall that connects the first opening and the second opening to form a through-hole, wherein the sidewall substantially surrounds the through-hole; and
a channel formed in the distal surface which, in use, receives a first portion of the catheter tube, wherein the channel is configured such that a distal edge of the first portion of the catheter tube is flush with or recessed from the distal surface when the first portion of the catheter tube is positioned in the channel,
wherein the fixation device further comprises a retainer configured to retain the first portion of the catheter tube in the channel,
wherein the body is configured such that the catheter tube extends in use from the channel through the through-hole to an exit point at which a second portion of the catheter tube exits the patient's skin, wherein the orientation of the first portion of the catheter tube is substantially different to an orientation of the second portion of the catheter tube, and
wherein the body is configured such that the sidewall is in use spaced from the exit point to create a window to a region of the patient's skin that substantially surrounds the exit point.

2. The fixation device according to claim 1, wherein the body comprises a channel wall forming the channel therein and wherein the channel wall is configured to act as the retainer.

3. The fixation device according to claim 1, wherein the body is configured such that a distal region of the channel forms a slit which extends from the distal surface to a proximal region of the channel, wherein the slit has a width that is smaller than a width of the first portion of the catheter tube configured to be received in the channel.

4. The fixation device according to claim 3, wherein the body is configured to form a lead-in to the slit to facilitate insertion of the catheter tube into the channel in use.

5. The fixation device according to claim 1, wherein the retainer comprises a clip.

6. (canceled)

7. (canceled)

8. The fixation device according to claim 1, wherein the sidewall is configured to form an overhang, wherein an end of the channel is provided in the overhang and wherein, when in use, the overhang spaces the second portion of the catheter tube from a proximal region of the sidewall.

9. The fixation device according to claim 1, wherein the first opening in the proximal surface is smaller than the second opening in the distal surface.

10. The fixation device according to claim 1, wherein the sidewall is shaped to form a chamber around the exit point in use.

11. (canceled)

12. The fixation device according to claim 1, wherein the body is configured to form a gap such that the second portion of the catheter tube can pass through the gap and into the through-hole when attaching the fixation device to the catheter tube in situ, wherein the gap extends from an end of the body to the side wall.

13. The fixation device according to claim 12, wherein the width of the gap is substantially equal to or less than the diameter of the catheter tube.

14. The fixation device according to claim 12, wherein the body is configured to be able to deform to an extent sufficient for a user to manually open the gap to pass the second portion of the catheter tube through the gap.

15. The fixation device according to claim 1, wherein the body further comprises one or more protrusions configured to grip a dressing positioned on or proximate the exit point.

16. The fixation device according to claim 1, wherein the body is configured such that, when viewed from a side in a direction substantially parallel to the proximal surface, the distal surface has a substantially convex-shaped envelope.

17. The fixation device according to claim 1, wherein the body is configured such that, when the first portion of the catheter tube is received in the channel in use, the catheter tube exits and extends away from the body in a substantially parallel direction to the patient's skin.

18. The fixation device according to claim 1, wherein the channel is a first channel which may, in use, receive a first portion of the catheter tube, and the body further comprises a second channel formed in the distal surface which, in use, may receive the first portion of the catheter tube as an alternative to the first portion of the catheter tube being received in the first channel, wherein the first channel is configured such that the catheter tube extends away from the body in a first direction when the first portion of the catheter tube is positioned in the first channel, and the second channel is configured such that the catheter tube extends away from the body in a second direction when the first portion of the catheter tube is positioned in the second channel, wherein the first direction is substantially different from the second direction.

19. The fixation device according to claim 18, wherein the body comprises a channel portion which is common to the first channel and to the second channel.

20. The fixation device according to claim 18, wherein the body is configured such that, when viewed in cross-section from a side in a direction substantially perpendicular to the proximal surface, the second channel comprises one or more curved regions which bend a third portion of the catheter tube.

21. The fixation device according to claim 1, wherein the body is configured such that, when viewed in cross-section from a side in a direction substantially parallel to the proximal surface, the channel comprises one or more curved regions which bend the first portion of the catheter.

22. The fixation device according to claim 1, wherein the fixation device further comprises a movable cover configured to cover at least a section of the window.

23. (canceled)

24. A catheter system for medical applications, wherein the catheter system comprises:
a catheter tube configured to be inserted into an exit point in a patient's skin; and
a fixation device according to claim 1.
